(19) United States
(12) Patent Application Publication
(10) Pub. No.: US 2024/0288412 A1
(43) Pub. Date: Aug. 29, 2024

(54) METHOD FOR THE (PARTIALLY) AUTOMATED ANALYSIS OF MINERAL OIL CONTAMINATION IN FOODSTUFFS DURING SAMPLE PREPARATION

(71) Applicant: AXEL SEMRAU GMBH, Sprockhövel (DE)

(72) Inventor: Marco NESTOLA, Sprockhövel (DE)

(21) Appl. No.: 18/697,329

(22) PCT Filed: Oct. 3, 2022
(86) PCT No.: PCT/EP2022/077445

§ 371 Date: Mar. 29, 2024

(30) Foreign Application Priority Data
Oct. 2, 2021 (DE) 102021125598.8

(51) Int. Cl. G01N 33/03

(52) U.S. Cl. CPC G01N 33/03

(57) ABSTRACT

Disclosed is a method for the (partially) automated analysis of mineral oil contaminants in foodstuffs comprising the following steps in sample preparation: contacting and dissolving resp. extracting the food with an excess of a solution of a hydrocarbon solvent and an alcohol at a temperature between 20° C. and the boiling point of the solvent mixture, after cooling and if an alcohol was used in the previous step, initiating the phase separation by adding water or a mixture of water and an alcohol, contacting the organic phase with an excess of at least 30% hydrogen peroxide and a compound of the general formula (I) R¹—COOH (I), in which R¹ represents H or the general partial formula (II) (R²)(R³)C(R⁴)— (II), where R², R³ and R⁴, independently, represent H, F, Cl, OH or methyl, in the presence of a small amount of a mineral acid at a temperature of above 20° C. to 70° C. for 5 min to 2 h with epoxidation, separation of the reagents by addition of an excess of water, use of the hydrocarbon solvent extract for the following analysis.

### CROSS-REFERENCE TO RELATED APPLICATIONS

This application claims priority to German application no. 102021125598.8, filed 2 Oct. 2021, the entire contents of which are incorporated herein by reference.

### FIELD OF THE INVENTION

The present invention relates to a method for analysis of mineral oil contaminants in foodstuffs.

### BACKGROUND OF THE INVENTION

Mineral oil contamination in the sense of the present invention refers to two different groups of chemical compounds found in mineral oil. MOSH (Mineral Oil Saturated Hydrocarbons) are saturated mineral oil hydrocarbons, mainly kerosenes and naphthenes, and MOAH (Mineral Oil Aromatic Hydrocarbons) are aromatic mineral oil hydrocarbons, mainly (polycyclic) aromatic hydrocarbons. While MOSH accumulate in the human body, MOAH are suspected of possibly containing carcinogenic substances.

Both types of contamination, which can originate from product packaging, lubricants or similar, have been routinely tested in foodstuffs for over 10 years and are the subject of constant discussion. The standard below and the DGF publication (German standard) explain the current state of the art regarding sample preparation and analysis.

In EN 16995—Vegetable oils and foodstuffs based on vegetable oils—Determination of MOSH and MOAH with online HPLC-GC-FID (as of June 2017), the direct measurement of MOSH and MOAH in oils/fats that are soluble in n-hexane is explained in section 9.1. In the presence of high amounts of biogenic n-alkanes, section 9.3 prescribes an additional preparative purification via a silica gel ALOX column for the determination of MOSH. In this case, MOAH remains on the chromatographic column and must subsequently be determined independently of MOSH in a separate analysis.

Furthermore, in section 9.4, for the determination of MOAH in the presence of significant amounts of biogenic olefinic sample components, it is proposed to perform a purification by epoxidation using chloroperbenzoic acid (CPBA), 77% purity, at room temperature under initial cooling. The achievable limits of quantitation for this test method are 10 mg per kilogram of food for MOSH and MOAH. Together with the manually error-prone sample preparation, this method is considered insufficient and unfit for practical use.

The German Standard Method for the Analysis of Fats, Fat Products, Surfactants and Related Substances—Method C-VI 22, 26th update edition, German Society for Fat Science 2020, hereinafter referred to as DGF 2020, describes on page 11 under point 7 a procedure for increasing the sensitivity by improved sample preparation (limit of determination of 1 mg/kg). For this purpose, the samples are further purified after saponification (step 7.2) via a silica gel column clean-up (step 7.4). Separation of biogenic n-alkanes via ALOX (step 7.3) is optional. Subsequently, the olefinic components are removed by epoxidation using chloroperbenzoic acid (CPBA), 77% purity, in ethanol at 40° C. and, if necessary, subsequent concentration of the solvent (step 7.5). This procedure also requires preparative column chromatography prior to the actual analysis in order to reliably determine MOSH and MOAH.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention is based on the task of providing a method for the (partially) automated analysis of mineral oil contamination in foodstuffs, including edible fats and oils, which is technically simpler than the pre-discussed methods from 2017 and 2020, in that it does not use column chromatography(s) already in sample preparation and further uses basic chemicals (such as hydrogen peroxide, formic acid, mineral acid) available in any laboratory for epoxidation instead of a special fine chemical that may be blind.

In a first aspect of the disclosure there is provided a method of preparing a sample for analysing mineral oil contamination in foodstuffs, wherein the method is at least partially automated, the method comprising:
(i) contacting and dissolving, or extracting, the foodstuff with an excess of a solution of hydrocarbon solvent and optionally an alcohol at a temperature of from about 20° C. to the boiling point of the solvent mixture;
(ii) cooling the solution, and if the alcohol was used in the previous step, initiating phase separation by addition of water or a mixture of water and alcohol;
(iii) contacting an organic phase comprising the dissolved or extracted foodstuff with an excess of at least 30% hydrogen peroxide and a compound of the general formula (I):

R¹—COOH (I);
in which R¹ represents H or the general partial formula (II)

(R²)(R³)C(R⁴)— (II)

wherein R², R³ and R⁴, independently of one another, are H, F, Cl, OH or methyl, in the presence of a catalytic amount of a mineral acid at a temperature of from about 20° C. to about 70° C. for about 5 min to about 2 h;
(iv) partitioning the solution by adding water to provide an aqueous phase and a reacted organic phase; and
(v) separating the reacted organic phase for analysis of mineral oil contamination in the foodstuff; and
(vi) optionally analysing the sample.

In embodiments, the ratio of hydrocarbon solvent to alcohol (if present) may be about 1:x (v/v), where x is the volume of alcohol relative to volume hydrocarbon solvent, and wherein x is a value from 0 to 10. This ratio may be expressed herein as (1:x, x: 0-10, v/v).

In embodiments, prior to the epoxidation step (iii), the method may further comprise evaporating the organic phase and adding an amount of halogenated aliphatic or aromatic hydrocarbon, wherein the amount is an amount necessary to dissolve the sample.

Alternatively in embodiments the epoxidation step (iii) may be conducted in the presence of a halogenated aliphatic or aromatic hydrocarbon and the method further comprises after step (iii) evaporating the organic phase and exchanging the solvent for a hydrocarbon solvent.

The hydrocarbon solvent may be any suitable hydrocarbon solvent capable of extracting mineral oil from a foodstuff. Accordingly, the hydrocarbon solvent is typically a non-polar hydrocarbon, such as a hydrocarbon comprising from 4 to 8 carbon atoms, preferably 5 or 6 carbon atoms. In embodiments, the hydrocarbon solvent is a hexane or a pentane, or a combination thereof. In embodiments the hydrocarbon solvent is selected from n-hexane, n-pentane, iso-pentane, iso-hexane, or a combination thereof. In embodiments, the hydrocarbon solvent is n-hexane. The hydrocarbon solvent may be used in step (i) optionally together with an alcohol. Any suitable alcohol may be used, however typically the alcohol is ethanol. In embodiments the ethanol is at least 96% vol ethanol. In embodiments the ethanol is absolute ethanol.

In embodiments there is provided the method as herein described wherein the method is fully automated.

The aforementioned epoxidation step (iii), generates the corresponding peracid from the compound of the general formula (I) in an acid-catalyzed manner in situ in the presence of hydrogen peroxide. As alkanoic acid, in addition to formic acid (R¹═H), also acetic acid or a substituted acetic acid can be used. Examples are, in addition to acetic acid, mono-, di-, or trifluoroacetic acid, mono-, di-, or trichloroacetic acid, glycolic acid, propionic acid, 2-methylpropanoic acid, neopentanoic acid. Hydrochloric, phosphoric, nitric and sulfuric acids can be used as mineral acids.

According to an embodiment of the present disclosure the foodstuff comprises alkaline hydrolyzable constituents in the organic phase and following step (i) and prior to step ii the alkaline hydrolyzable constituents are saponified by:
contacting the dissolved or extracted foodstuff with an excess of a concentrated aqueous alkali metal hydroxide solution or other strong base above about 20° C. to about 70° C., and
extracting the non-saponified constituents by adding a hydrocarbon solvent; and
optionally repeating the extraction with the hydrocarbon solvent one or more times.

In embodiments the alkali metal hydroxide solution is a sodium or potassium hydroxide solution, and the other strong base is an alkaline earth hydroxide such as barium, calcium and strontium hydroxide or an organic base such as an alkylamine.

In embodiments there is provided the method as herein described wherein the method does not involve a clean-up step prior on silica-gel to remove the matrix prior to the epoxidation step.

The methods may be carried out on any suitable determined amount of sample. Beginning the process with a known amount of sample is important for quantifying the mineral oil content. In some instances, the methods of the invention allow for analysis of smaller amounts of sample due to their improved detection of MOSH/MOAH contaminants, however sufficient sample is required to ensure the amount of MOSH/MOAH contaminant, if present, would be above the detection limit of the analytical technique selected (e.g LC-GC-FID). In some embodiments, a maximum sample of not more than about 10 millilitres (mL), about 9 mL, about 8 mL about 7 mL, about 6 mL, about 5 mL, about 4 mL, about 3 mL, about 2 mL or about 1 millilitre (mL) may be used. In embodiments the minimum sample volume may be at least about 1 microlitre (μL), about 10 μL, about 50 μL, about 75 μL, about 100 μL, about 150 μL, about 200 μL, about 300 μL, about 400 μL, about 500 μL, about 600 μL, about 700 μL, about 800 μL, about 900 μL or about 950 μL. In embodiments the sample volume may be from any of these minimum volumes to any of these maximum volumes, for example from about 1 μL to about 10 mL or about 500 μL to about 2 mL. The skilled person will be able to select appropriate amounts of reagents and/or solvents depending on the mass/volume of the initial sample aliquot.

According to an embodiment of the present disclosure, the foodstuff comprises acidic hydrolyzable constituents in the organic phase and following step (i) and prior to step (ii) the acid hydrolyzable constituents are hydrolyzed by:
contacting the dissolved or extracted foodstuff with an excess of a concentrated aqueous hydrochloric acid solution or other strong acid at about ° C. to about 70° C.; and
extracting the non-hydrolyzed constituents by adding a hydrocarbon solvent; and
optionally repeating the extraction with a hydrocarbon solvent one or more times.

In embodiments the other strong acid is a mineral acid such as phosphoric acid, sulfuric acid or nitric acid.

According to an embodiment of the present disclosure the food stuff is a dried foodstuff or is a foodstuff present in an aqueous phase and, prior to step (i) the mineral oil contaminations are extracted from the foodstuff by
contacting the foodstuff with an excess of an organic water-insoluble solvent; and
separating the solvent comprising extracted mineral oil impurities from the foodstuff using suitable means.

In an embodiment the extraction of the mineral oil components from the foodstuff is carried out with an excess of a (1:x, x:0-10, v/v) solution of hydrocarbon solvent and an alcohol at a temperature or from about 20° C. to the boiling point of the solvent mixture.

In embodiments the alcohol is ethanol. In embodiments the ethanol is at least 96% vol ethanol. In embodiments the ethanol is absolute ethanol.

Preferably, any solvent and/or reagent used in the methods described herein is free of MOSH/MOAH contamination.

The MOSH/MOAH content of the solvent and/or regents may be determined by a similar method as described for the methods of the present disclosure, such as gas chromatography (e.g. using a MXT-1 column—Siltek-treated stainless steel, 15 m×0.25 mm×0.25 μm, Restek, Bellefonte, PA, USA) or any other suitable technique including the for example LC-GC-FID as described herein. Accordingly, the MOSH/MOAH content of any solvent and/or regent used in the methods described herein may be below the detection limit of the analytical technique intended to be used to analyse the MOSH/MOAH content of the foodstuff sample.

In some embodiments the methods comprise an initial step of determining the MOSH/MOAH content of the solvents and reagents prior to their use, and optionally purifying the solvent and/or regent to remove MOSH/MOAH content prior to their use in the methods of the present disclosure. The solvent and/or reagent may be purified by techniques known in the art, including distillation, chromatography, and so on.

In an embodiment of the present disclosure, the saponification of the organic phase is carried out with an excess of a concentrated alkali metal hydroxide solution in the form of sodium hydroxide solution and/or potassium hydroxide solution at from about 20° C. to about 70° C.

In an embodiment of the present disclosure, the halogenated hydrocarbon chloroform is added to the organic phase prior to the epoxidation step (iii).

In an embodiment of the present invention, the epoxidation, step (iii) comprises
contacting the organic phase with a mixture of 50% hydrogen peroxide and concentrated formic acid and/or acetic acid in the presence of a catalytic amount of concentrated phosphoric acid and/or sulfuric acid at a temperature of from about 20° C. to about 70° C. for about 10 min to about 60 min.

According to an embodiment of the present disclosure, the foodstuff is selected from natural fats, protein-containing foodstuffs, carbohydrate-rich foodstuffs, alcohol-containing foodstuffs, alkaloid-containing foodstuffs, vegetables and vegetable products, fruit and fruit products, spices or herbs, drinking water, soft drinks, functional foodstuffs, food supplements, dietary foodstuffs, novel foods, vitamins, minerals, enzymes, lipids, amino acids, additives or mixtures of the aforementioned substances and/or products of the foodstuff classes. According to an embodiment of the present disclosure the natural fat is a fruit pulp fata seed fat, or an animal fat or a combination thereof.

According to another embodiment of the present disclosure, the fat-containing food product is milk or a milk product, chocolate, chocolate products, cocoa, margarine products, mixed fat products, infant formula and follow-on formula, or a mixture of the aforementioned products.

In a second aspect of the present disclosure there is provided a system, comprising:
a receptacle for a sample of foodstuff for analysis of its mineral oil content, the receptacle in fluid communication with a first well, a second well, a third well and a fourth well,
the first well adapted for the introduction into the receptacle of a hydrocarbon solvent and optionally an alcohol at a temperature of from about 20° C. to the boiling point of the solvent mixture,
the second well adapted for the introduction to the receptacle of an excess of at least 30% hydrogen peroxide
a third well adapted for the introduction to the receptacle of a compound of the general formula (I):

R¹—COOH (I);
in which R¹ represents H or the general partial formula (II)

(R²)(R³)C(R⁴)— (II)
wherein R², R³ and R⁴, independently of one another, are H, F, Cl, OH or methyl, in the presence of a catalytic amount of a mineral acid at a temperature of from about 20° C. to about 70° C.
the fourth well adapted to introduce water into the receptacle
a separator to separate a reacted organic phase from the receptacle and transporting the reacted organic phase to an analyser
a controller for controlling the addition to the receptacle of the solvent contained in each of the first, second, third and fourth wells, and also for controlling the temperature of the receptacle; and
optionally the analyser capable of detecting the mineral oil in the reacted organic phase.

In some embodiments the system comprises a shaker to shake the receptacle, wherein the shaker may also be under the control of the controller. In some embodiments the system may be adapted to automatically transfer the receptacle to the shaker.

In some embodiments the separator comprises a centrifuge. The centrifuge may be controlled by the controller. In some embodiments the system may be adapted to automatically transfer the receptacle to the centrifuge.

In a third aspect of the present disclosure there is provided use of the system as herein described to conduct the method as herein described.

In a further aspect there is provided the MOSH and MOAH analysis of a sample wherein the sample is prepared according to the methods and embodiments of the first aspect as herein described and the sample is analysed.

In a further aspect there is provided the MOSH and MOAH analysis of a sample wherein the sample is prepared using the system of the second aspect and embodiments as herein described and analysed.

In embodiments the sample is analysed by LC-GC-FID or the sample is pre-separated using HPLC and analysed using GC-MS or GCXGC-MS.

As used herein, except where the context requires otherwise, the term “comprise” and variations of the term, such as “comprising”, “comprises” and “comprised”, are not intended to exclude further additives, components, integers or steps.

It will be understood that where an embodiment is defined as “comprising”, the narrower position of “consists essentially of” and “consists of” are also disclosed. The term “consists essentially of” or variations such as “consisting essentially of” denotes that the embodiment includes all listed components or steps and may include other non-listed components or steps that do not materially affect the basic properties or function of the embodiment. The term “consists of” or variations such as “consisting of” is intended to denote that the embodiment is defined to exclude further additives, components or steps.

Further aspects of the present disclosure and further embodiments of the aspects described in the preceding paragraphs will become apparent from the following description, given by way of example and with reference to the accompanying drawings.

### BRIEF DESCRIPTION OF THE DRAWINGS

FIG. 1 shows a LC-GC-FID MOAH chromatograms of palm oil (RBD—refined, bleached, deodorized) after epoxidation according to DGF standard method and EN 16995:2017.

FIG. 2 shows an LC-GC-FID chromatogram of the MOAH fraction of a palm olein after sample preparation according to the invention, example 1, (uppermost chromatogram) and the prior art, comparative example [EN 16995, DGF-2020]. In the right panel: time less than 20 min.

FIG. 3 shows an LC-GC chromatogram of the MOAH fraction of a 1:1 mixture of olive and sunflower oil, after sample preparation to which a mineral oil has been added to simulate contamination. The upper chromatogram shows a preparation according to DGF 2020, comparative example, and the lower curve shows the method according to the invention, example 2.

FIG. 4 shows an LC-GC-FID MOAH chromatogram overlay of epoxidized RBD palm oil showing the residual polyunsaturates in dependence of the used epoxidation technique.

FIG. 5 shows a MOAH chromatogram of EIE palm oil after epoxidation highlighting a peak cluster found in multiple refined palm oil fractions.

FIG. 6 shows an exploded view of the highlighted peak cluster from FIG. 5. Monocyclic aromatic steroid hydrocarbons tendentially identified in refined palm oils according to mass spectra and elution order from literature.

FIGS. 7 A-C show obtained mass spectra for monocyclic aromatic hydrocarbons derived from sitosterol.

FIG. 8 shows a drawing of aromatic hydrocarbon structures and their half-life times for epoxidation with performic acid in chloroform.

FIG. 9 shows a workflow for MOSH/MOAH sample preparation and analysis.

FIGS. 10 A and 10 B show LC-GC-FID chromatograms of a reagent blank for MOSH (10A) and MOAH (10B).

FIGS. 11 A-F show LC-GC-FID MOAH chromatograms of samples of cocoa butter (A), sunflower oil (B), spiked rapeseed oil (C), spiked olive oil (D), spiked sunflower oil (E) and spiked palm oil (F) from a trial during development of DGF standard method C-VI 22.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

### Definitions

For purposes of interpreting this specification, terms used in the singular will also include the plural and vice versa. For example, “a” means one or more unless indicated otherwise.

Unless defined otherwise, all technical and scientific terms used herein have the same meanings as commonly understood by one of ordinary skill in the art to which this disclosure belongs. Although any materials and methods similar or equivalent to those described herein can be used to practice or test the present disclosure, the preferred materials and methods are now described.

One skilled in the art will recognise many methods and materials similar or equivalent to those described herein, which could be used in the practice of the present disclosure. The present disclosure is in no way limited to the methods and materials described.

The term “measurement” used in the specification should be construed in its broadest sense, including quantitative and qualitative measurement. Unless otherwise stated it should be understood that measurements are conducted at ambient conditions, typically room temperature and pressure, for example about 20-25° C. at about 1 atmosphere (atm).

As used herein, the term “and/or” means “and”, or “or”, or both.

The term “(s)” following a noun contemplates the singular and plural form, or both.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9, and 10) and also any range of rational numbers within that range (for example, 2 to 8, 1.5 to 5.5, and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

Various features of the invention are described with reference to a certain value, or range of values. These values are intended to relate to the results of the various appropriate measurement techniques, and therefore should be interpreted as including a margin of error inherent in any particular measurement technique. Some of the values referred to herein are denoted by the term “about” to at least in part account for this variability. The term “about”, when used to describe a value, may mean an amount within ±10%, ±5%, ±1% or ±0.1% of that value.

As used herein the term “excess”, unless the context requires otherwise, is intended to mean either an excess by volume or a molar excess. Where it is not clear based on context, references herein to an excess should be interpreted as an excess by volume.

### Foodstuff

According to an embodiment of the present disclosure, the foodstuffs are selected from natural fats, protein-containing foodstuffs, carbohydrate-rich foodstuffs, alcohol-containing foodstuffs, alkaloid-containing foodstuffs, vegetables and vegetable products, fruit and fruit products, spices or herbs, drinking water, soft drinks, functional foodstuffs, food supplements, dietary foodstuffs, novel foods, vitamins, minerals, enzymes, lipids, amino acids, additives or mixtures of the aforementioned substances and/or products of the food classes. Examples of protein foods are meat, meat products including sausage, meat extract, broth seasoning, gelatin, fish, crustaceans, shellfish and mollusks, fish products, eggs, egg products, vegetable protein products including those from soy, lupins. Examples of carbohydrate-rich foods include sugar, sugar alcohols, sugar confectionery, honey, cereals (grains), bread and bakery products, baking agents, baking powder, pasta, starch. Examples of alcohol-containing foods are wine, sparkling wine, beer, brandy, as an alcoholic additive in confectionery and baked goods. Examples of alkaloid-containing foods are coffee, tea, cocoa and chocolate. Examples of vegetables and vegetable products are fresh vegetables such as potatoes, tomatoes, cabbage vegetables, legumes, mushrooms, as well as vegetable durables such as frozen vegetables, canned vegetables, dried vegetables, fermented vegetables, pickled vegetables. Examples of fruit and fruit products include fresh fruit, dried fruit, candied fruit, jams, jellies and marmalades, fruit juices and fruit nectars. Examples of spices or herbs, in addition to fresh produce, include fruit, seed, flower, root, bark, leaf and herb spices, spice blends, soy sauce, essences, table salt, vinegar and fruit acids. Examples of soft drinks are mineral water, sweet soft drinks, non-alcoholic soft drinks, sodas and isotonic drinks. Examples of functional foods are products fortified with omega-3 fatty acids, ACE vitamins or beta-glucan. Examples of additives are antioxidants, emulsifiers, thickeners, stabilizers, humectants and flavorings.

According to an embodiment of the present disclosure, a natural fat, in particular a refined fat, is understood to be a foodstuff within the meaning of the present invention, such as vegetable fats, for example, a fruit pulp fat, for example, palm oil, olive oil, avocado oil, a seed fat, for example, coconut fat, salt fat, palm kernel fat, babassu fat, bay leaf fat, nutmeg butter, ucuhuba fat, dika fat, cocoa butter, Shea butter, Borneo tallow, cottonseed oil, kapok oil, okra oil, cone seed oil, pumpkin seed oil, corn germ oil, grain oils, sunflower oil, sesame oil, linseed oil, perilla oil, hemp oil, tea seed oil, safflower oil, niger oil, grape seed oil, poppy seed oil, beechnut oil, hazelnut oil, soybean oil, peanut oil, beet oil, Chinese wood oil, oiticica oil, boleko oil, parinarium oils, ricinus oil, chaulmoogra oil, hydnocarpus oil, gorli oil, vernonia oil. Further, a fat, in particular a purified fat, within the meaning of the present invention is understood to be an animal fat such as a land animal fat, for example, pig fat, beef tallow, mutton tallow, horse fat, goose fat, chicken fat or a sea animal oil, for example, whale oil, fish oils, fish and whale liver oils, sperm oil. A mixture of the aforementioned fats is also possible here. Preferred fats in the sense of the present invention are all marketable refining stages of palm, shea, coconut and salt fats as well as edible oils and edible fats, which include by way of example, in addition to vegetable fats and oils, butter, margarine, special margarine. Special fats (plate fats, deep-frying fats), separating oils, mayonnaise and salad dressings.

Generic detection of MOSH/MOAH content of foods is difficult in certain oils due to the presence of other components in the oil with overlapping signal to MOSH/MOAH, for example polyunsaturates. In particular palm oil is known to have a difficult matrix which makes detection of MOSH/MOAH content challenging. Surprisingly the inventors have found that the methods of the present disclosure are able to detect MOSH/MOAH content in food comprising palm oil with increased sensitivity.

In some embodiments the foodstuff comprises palm oil.

According to another embodiment of the present disclosure, the fat-containing food product is milk or a milk product, chocolate, chocolate products, cocoa, margarine products, mixed fat products, infant formula and follow-on formula, or a mixture of the aforementioned products. Dairy products are, for example, condensed milk, dried milk products, sour milk products, cream, milk-based dessert products, butter, cheese, ice cream. Chocolate products are for example praline, drinking chocolate, chocolate powder, hollow chocolate, chocolate sprinkles. Margarine products are for example household margarine, half-fat margarine, baking margarine. Infant formula and follow-on formula is for example infant formula in dry form based on dry milk powder.

### Automation

As used herein the phrase “at least partially automated” refers to a process or method in which at least two or more (or at most all) of the steps of the process or method may be carried out automatically without requiring manual handling.

In embodiments partially automated refers to 2 automated steps. In embodiments partially automated refers to 3 automated steps, or 4 automated steps, or 5 automated steps, or 6 automated steps. In embodiments partially automated refers to all but 1 steps are automated, or all but 2 steps are automated, or all but 3 steps are automated, or all but 5 steps are automated, or all but 6 steps are automated.

In embodiments some of the automated steps are sequential steps. In embodiments a sequence of automated steps may be followed by a manual handling step, which is followed by two or more further automated steps.

As used herein the phrase “fully automated process” refers to a process in which none of the steps require manual human handling other than providing a sample at the beginning of the process.

As described herein the disclosure relates to an at least partially automated method of analysing the MOAH and MOSH content in mineral oils, preferably food oils.

### Pre-Extraction

Optionally the method may be applied when the foodstuff is a dried foodstuff and a pre-extraction step to obtain the food oil is required. This may be done by suitable means known in the art.

Suitable means include solid-liquid extraction in a Soxhlet. Likewise, the common methods for fat extraction according to Weibull-Stoldt, Röse-Gottlieb or Schmid-Bondzynski-Ratzlaff are suitable, depending on the sample matrix, for extracting the non-polar mineral oil components.

### Extraction Step

In embodiments of the disclosure the method includes an extraction step.

The extraction step may comprise:
(i) contacting and dissolving, or extracting, the foodstuff with an excess of a solution of a hydrocarbon solvent and optionally an alcohol at a temperature of from about 20° C. to the boiling point of the solvent mixture; and
(ii) cooling the solution.

In embodiments if an alcohol was used in step (i) the method further comprises initiating phase separation by addition of water or a mixture of water and the alcohol.

In embodiments the vol:vol ratio of hydrocarbon solvent to alcohol may be from 1:x, wherein x is the volume of alcohol relative to hydrocarbon solvent, wherein x is the value of from 0 to 10.

### Saponification

According to an embodiment of the present disclosure the foodstuff comprises alkaline hydrolyzable constituents in the organic phase and following step (i) and prior to step ii the alkaline hydrolyzable constituents are saponified by:
contacting the dissolved or extracted foodstuff with an excess of a concentrated aqueous alkali metal hydroxide solution or other strong base above about 20° C. to about 70° C., and
extracting the non-saponified constituents by adding a hydrocarbon solvent; and
optionally repeating the extraction with the hydrocarbon solvent one or more times.

In embodiments the extraction with the hydrocarbon solvent is repeated from 1 to 10 times.

In embodiments the extraction step is repeated 1, or 2, or 3, or 4, or 5, or 6, or 7, or 8, or 9, or 10 times.

The hydrocarbon solvent may be any suitable hydrocarbon solvent capable of extracting mineral oil from a foodstuff. Accordingly, the hydrocarbon solvent is typically a non-polar hydrocarbon, such as a hydrocarbon comprising from 4 to 8 carbon atoms, preferably 5 or 6 carbon atoms. In embodiments, the hydrocarbon solvent is a hexane or a pentane, or a combination thereof. In embodiments the hydrocarbon solvent is selected from n-hexane, n-pentane, iso-pentane, iso-hexane, or a combination thereof. In embodiments, the hydrocarbon solvent is n-hexane. The saponification step, when required may be performed after the extraction solution is cooled (step (ii) as described herein).

In embodiments the alkali metal hydroxide solution is a sodium or potassium hydroxide solution.

In embodiments the other strong base is an alkaline earth hydroxides such as barium, calcium and strontium hydroxide or organic bases such as alkylamines.

In embodiments the saponification of the organic phase is carried out with an excess of a concentrated alkali metal hydroxide solution in the form of sodium hydroxide solution and/or potassium hydroxide solution at a temperature from about 20° C. to about 70° C.

Advantageously the saponification step is not limited by sample volume and can be scaled as required for the sample. One reason the sample volume may be increased is to increase sensitivity of the measurement.

In embodiments the method does not involve a clean-up step on silica-gel to remove the matrix prior to the epoxidation step.

In prior art MOSH/MOAH analytics, HPLC clean-up on bare silica gel served to remove the matrix, this is impractical when the sample amount is increased beyond the limits of the used HPLC column. Choosing a bigger HPLC column leads to problems regarding the transfer volume into GC. Advantageously, saponification can handle larger sample volumes. Conveniently, this step can also be automated.

### Acid Hydrolysis

According to an embodiment of the present disclosure, the foodstuff comprises acidic hydrolyzable constituents in the organic phase and following step (i) and prior to step ii the acid hydrolyzable constituents are hydrolyzed by:
contacting the dissolved or extracted foodstuff with an excess of a concentrated aqueous hydrochloric acid solution or other strong acid at about 20° C. to about 70° C.; and
extracting the non-hydrolyzed constituents by adding a hydrocarbon solvent; and
optionally repeating the extraction with the hydrocarbon solvent one or more times.

In embodiments the extraction with the hydrocarbon solvent is repeated from 1 to 10 times.

In embodiments the extraction step is repeated 1, or 2, or 3, or 4, or 5, or 6, or 7, or 8, or 9, or 10 times.

The hydrocarbon solvent may be any suitable hydrocarbon solvent capable of extracting mineral oil from a foodstuff. Accordingly, the hydrocarbon solvent is typically a non-polar hydrocarbon, such as a hydrocarbon comprising from 4 to 8 carbon atoms, preferably 5 or 6 carbon atoms. In embodiments, the hydrocarbon solvent is a hexane or a pentane, or a combination thereof. In embodiments the hydrocarbon solvent is selected from n-hexane, n-pentane, iso-pentane, iso-hexane, or a combination thereof. In embodiments, the hydrocarbon solvent is n-hexane.

In embodiments the other strong acid may be a mineral acid such as phosphoric acid, sulfuric acid or nitric acid.

### Epoxidation Step

In embodiments the method comprises an epoxidation step. The epoxidation step comprising:
contacting an organic phase comprising the dissolved or extracted foodstuff with an excess of at least 30% hydrogen peroxide and a compound of the general formula (I):

R¹—COOH (I);
in which R¹ represents H or the general partial formula (II)

(R²)(R³)C(R⁴)— (II),
wherein R², R³ and R⁴, independently of one another, are H, F, Cl, OH or methyl, in the presence of a catalytic amount of a mineral acid at a temperature of from about 20° C. to about 70° C. for about 5 min to about 2 h;
partitioning the solution by adding water to provide an aqueous phase and a reacted organic phase, and
separating the reacted organic phase for analysis of mineral oil contamination in the foodstuff.

The hydrocarbon solvent may be any suitable hydrocarbon solvent capable of extracting mineral oil from a foodstuff. Accordingly, the hydrocarbon solvent is typically a non-polar hydrocarbon, such as a hydrocarbon comprising from 4 to 8 carbon atoms, preferably 5 or 6 carbon atoms. In embodiments, the hydrocarbon solvent is a hexane or a pentane, or a combination thereof. In embodiments the hydrocarbon solvent is selected from n-hexane, n-pentane, iso-pentane, iso-hexane, or a combination thereof. In embodiments, the hydrocarbon solvent is n-hexane.

In embodiments, the epoxidation step is conducted in the presence of a halogenated aliphatic or aromatic hydrocarbon.

In embodiments prior to the epoxidation step, the method comprises evaporating the organic phase and adding a halogenated aliphatic or aromatic hydrocarbon. The amount of halogenated aliphatic or aromatic solvent may be an amount necessary to dissolve the sample.

When the epoxidation step (iii) is conducted in the presence of a halogenated aliphatic or aromatic hydrocarbon, the method may further comprise evaporating the halogenated aliphatic or aromatic hydrocarbon and addition of a hydrocarbon solvent following epoxidation, to exchange the halogenated solvent for a hydrocarbon solvent preferred for analysis. Any of the hydrocarbon solvents described for the sample preparation step may be used, however, typically the hydrocarbon solvent is hexane.

In embodiments, solvent exchange is performed by any suitable means known in the art, for example applying vacuum or a gas stream along with heat and/or shaking, or combinations thereof.

In embodiments the halogenated aliphatic hydrocarbon may be (but is not limited to) chlorinated aliphatic hydrocarbons such as: chloromethanes (monochloromethane, dichloromethane, chloroform and carbon tetrachloride), chloroethanes, vinyl chloride, 1,1-dichloroethylene, 1,2-dichloroethylene, trichloroethylene and perchloroethylene, chloropropanes, chlorobutanes, chlorobutenes, fluorinated aliphatic fluoromethanes, fluoroethanes, fluoroethylenes, fluoropropanes, fluorobutanes, and fluorobutenes.

In a preferred embodiment the halogenated aliphatic hydrocarbon is a chloromethane, preferably dichloromethane or chloroform, most preferably chloroform.

In embodiments, the halogenated aromatic hydrocarbon may be (but is not limited to) chlorinated aromatic hydrocarbons such as chlorobenzenes including, monochlorobenzene, dichlorobenzene, trichlorobenzene, tetrachlorobenzene, pentachlorobenzene, and hexachlorobenzene and fluorinated aromatic hydrocarbons such as fluorobenzenes including, monofluorobenzene, difluorobenzene, trifluorobenzene, tetrafluorobenzene, pentafluorobenzene, and hexafluorobenzene.

In embodiments, the solution of halogenated aliphatic or aromatic hydrocarbon does not comprise meta-chloroperoxybenzoic acid (mCPBA).

In some embodiments, the organic phase comprising the extracted or dissolved foodstuff further comprises a keeper solvent. The keeper solvent preferably does not comprise any reactive unsaturated C—C bonds (eg olefins) to the epoxidation conditions. The keeper solvent is intended to keep the components of the foodstuff in solution to assist solvent exchange steps, and is therefore preferable in methods involving, for example, a solvent exchange between the hydrocarbon solvent and the halogenated solvent. Any suitable keeper solvent unreactive to the epoxidation conditions may be used. Suitable keeper solvents include sebacate and the like. The amount of keeper solvent is not limited provided a minimum amount is present to retain the extracted or dissolved foodstuff in solution, which will depend on the specifics of the extracted or dissolved foodstuff.

According to a preferred embodiment of the present invention, epoxidation is carried out by contacting the organic phase with a mixture of about 50% hydrogen peroxide and concentrated formic acid and/or acetic acid in the presence of a catalytic amount of concentrated phosphoric acid and/or sulfuric acid at a temperature of about 20° C. to about 70° C. for from about 10 min to about 60 min.

In embodiments epoxidation is carried out by contacting the organic phase with a mixture of about 50% hydrogen peroxide and concentrated formic acid and/or acetic acid in the presence of a catalytic amount of concentrated sulfuric acid at a temperature of about 20° C. to about 70° C. for from about 10 min to about 60 min.

In embodiments the epoxidation reaction is carried out in the absence of phosphoric acid.

In embodiments the formic acid is a concentrated formic acid. In embodiments the formic acid is neat formic acid (eg 100% concentration). Importantly the acid should not contain any MOSH/MOAH impurities.

In embodiments acetic acid is a concentrated acetic acid. In embodiments the formic acid is neat formic acid (eg 100% concentration). Importantly the acid should not contain any MOSH/MOAH impurities.

As used herein ‘catalytic amount’ as used in relation to an acid present in the epoxidation reaction step means an amount sufficient to increase the reaction rate, for example in an amount of from about 5% to about 10%.

In embodiments the concentrated sulfuric acid has a purity of at least 95%.

In embodiments the concentrated phosphoric acid has a purity of at least 85%

In embodiments the catalytic amount of concentrated phosphoric acid and/or sulfuric acid is from about 5% to about 10%. In preferred embodiments the catalytic amount is about 10%.

In embodiments, the epoxidation is carried out in the presence of a catalytic amount of concentrated sulfuric acid. In embodiments, the epoxidation reaction does not comprise phosphoric acid.

The temperature of the epoxidation reaction will vary depending on the analyte (size, chemical composition, etc.). In some embodiments, the minimum temperature for the epoxidation reaction may be at least about 20° C., about 25° C., about 30° C., about 35° C., about 40° C., about 45° C., or about 50° C. In some embodiments, the maximum temperature of the epoxidation reaction may be not more than about 70° C., about 65° C., about 60° C., about 55° C., or about 50° C. The temperature of the epoxidation step may be from any of these minimum temperatures to any of these maximum temperatures, provided that the maximum temperature is greater than the minimum temperature. For example, in some embodiments, the temperature of the epoxidation step is from about 30° C. to about 55° C. or from about 40° C. to about 60° C.

The time of reaction required will in part depend on the temperature selected and the nature of the analyte and the presence of biogenic interferences. Typically, the epoxidation reaction is allowed to run from about 10 min to about 60 min.

In embodiments the sample volume for analysis may be up to 400 μL. Advantageously increasing the sample volume increases the sensitivity of the analysis. Typically prior art methods are limited to maximum 100 μL sample volumes.

In embodiments the hydrogen peroxide used in the epoxidation step, step (iii), is at least 35% hydrogen peroxide, or at least 40% hydrogen peroxide, or at least 45% hydrogen peroxide, or at least 50% hydrogen peroxide.

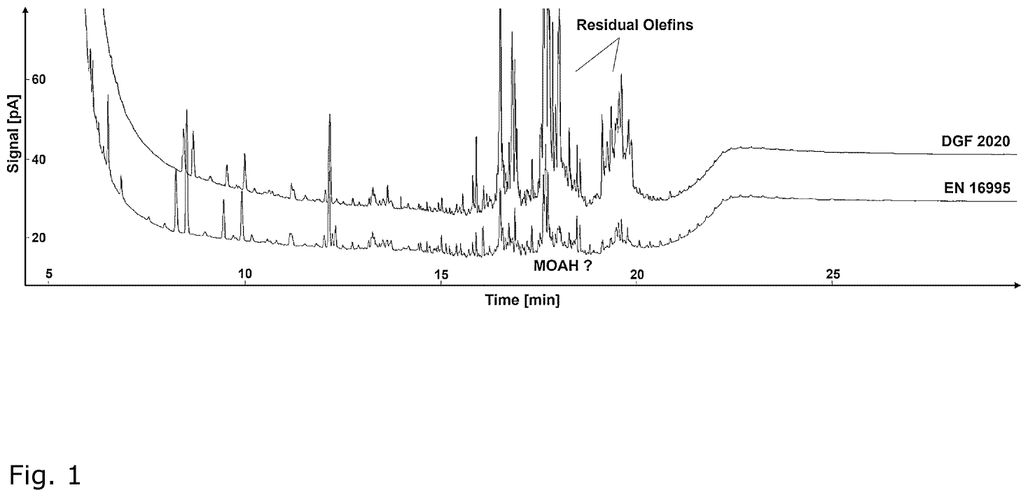

One issue with the prior art methods of MOSH/MOAH analysis is the introduction of residual biogenic interferences after the epoxidation step using mCPBA. These contaminants hinder the identification and determination of MOAH (see FIG. 1). Advantageously the method of the present disclosure ameliorates these effects allowing a better identification of MOAH in the sample.

### Separation Step

The method of the present disclosure further comprises a separation step of
partitioning the solution by adding water to provide an aqueous phase and a reacted organic phase, and
separating the reacted organic phase for analysis of mineral oil contamination in the foodstuff.

### Subsequent Analysis

The methods of the present disclosure relate to a method of preparing a sample for the analysis of mineral oil contaminants.

The prepared sample may be analysed by suitable means known in the art, for example LC-GC-FID (Online coupled Liquid Chromatography Gas Chromatography Flame Ionisation detection). Alternatively, the obtained extract is pre-separated using HPLC and then analysed with GC-MS or GCXGC-MS.

In embodiments, the sample is evaporated to dryness before addition of an aliquot of n-hexane. Current calibration curves for MOSH/MOAH have been developed for n-hexane samples, so in preferred methods any alternative solvent may be removed and exchanged for n-hexane prior to analysis. The evaporation may be achieved by any of the solvent exchange techniques known in the art and/or described herein. In the at least partially automated methods of the present disclosure, this may be carried out in an autosampler to also automate this step.

It will be appreciated that when using the analytical methods as described herein a more precise MOSH/MOAH content may be determined and on the basis of these results whether the sale of the foodstuff is recommended based on the recommended MOSH/MOAH limits for foodstuff.

In embodiments there is provided a method of preparing a sample as herein described and analysing the sample using LC-GC-FID.

### System

Also described herein are systems for carrying out the partially automated methods for analysing mineral oil contaminants in foodstuffs described herein.

Accordingly, the second aspect provides a system, comprising:
a receptacle for a sample of foodstuff for analysis of its mineral oil content, the receptacle in fluid communication with a first well, a second well, a third well and a fourth well,
the first well adapted for the introduction into the receptacle of a hydrocarbon solvent and optionally an alcohol at a temperature of from about 20° C. to the boiling point of the solvent mixture,
the second well adapted for the introduction to the receptacle of an excess of at least 30% hydrogen peroxide
a third well adapted for the introduction to the receptacle of a compound of the general formula (I):

R¹—COOH (I);
in which R¹ represents H or the general partial formula (II)

(R²)(R³)C(R⁴)— (II),
wherein R², R³ and R⁴, independently of one another, are H, F, Cl, OH or methyl, in the presence of a catalytic amount of a mineral acid at a temperature of from about 20° C. to about 70° C.
the fourth well adapted to introduce water into the receptacle
a separator to separate a reacted organic phase from the receptacle and transporting the reacted organic phase to an analyser
a controller for controlling the addition to the receptacle of the solvent contained in each of the first, second, third and fourth wells, and also for controlling the temperature of the receptacle; and
optionally the analyser capable of detecting the mineral oil in the reacted organic phase.

In embodiments, the system may further comprise a fifth well adapted to introduce to the receptacle a catalytic amount of a mineral acid. Typically the mineral acid is added together with the compound of the general formula (I), however in some embodiments, the system comprises this optional fifth well for its separate addition. Separate addition of the catalytic acid may assist control the epoxidation reaction conditions within the system.

In embodiments, the system may further comprise a sixth well adapted to introduce a halogenated aliphatic or aromatic hydrocarbon to the receptacle prior to the addition of hydrogen peroxide and/or compound of the general formula (I).

In some embodiments, the system comprises a seventh well for supplying alcohol to the receptacle separately to the hydrocarbon solvent.

In embodiments, the first, second, third, and fourth, and optional fifth, sixth and seventh, wells each comprise an inlet (eg the first well comprises a first inlet, the second well comprises a second inlet and the third well comprises a third inlet, the fourth well comprises a fourth inlet, the fifth well comprises a fifth inlet, the sixth well comprises a sixth inlet and the seventh well comprises a seventh inlet) for charging each well with the required solvent and/or reagent. Each inlet may be adapted for manual charging of a desired volume of solvent, or may be in communication with an external supply of required solvent and/or reagent. The supply of each solvent and/or reagent from each well to the receptacle is controlled by the controller. To facilitate this, each well may comprise a pump to control the supply of its contents to the receptacle. The pump may be a metered pump capable of supplying a desired amount of solvent and/or reagent to the receptacle or in embodiments where a well is charged with a fixed amount of solvent and/or reagent the pump may be operable to discharge the entire contents of the well to the receptacle.

The receptacle may be adapted to control the temperature of its contents. Accordingly, in embodiments, the receptacle may comprise a thermocouple. In other embodiments, the receptacle comprises an inlet and outlet capable of transporting the receptacle contents through a heat exchanger to control the temperature of the contents of the receptacle. The heat exchanger may be adapted to heat and/or cool the receptacle contents.

In some embodiments the system comprises a shaker to shake the receptacle, wherein the shaker may also be controllable by the controller. In some embodiments the system may be adapted to automatically transfer the receptacle to the shaker.

In some embodiments the separator comprises a centrifuge. The centrifuge may be controlled by the controller. In some embodiments the system may be adapted to automatically transfer the receptacle to the centrifuge.

In some embodiments, the separator comprises an autosampler for transporting the reacted organic phase to the analyser.

In some embodiments, the system further comprises a solvent evaporation unit. A solvent evaporation unit is preferred when the methods include the step swapping a hydrocarbon solvent, and a halogenated aliphatic hydrocarbon or halogenated aromatic hydrocarbon solvent. The solvent evaporation unit may be any suitable for removing the hydrocarbon solvent (eg hexane and/or pentane) selectively from the compounds present in the sample, and/or selectively for removing the halogenated aliphatic or aromatic hydrocarbon from the compounds present in the sample. The solvent evaporation unit may therefore be one in which the hydrocarbon solvent is removed under reduced pressure or a gas stream (eg nitrogen or argon), optionally while applying heat and/or agitation (eg shaking or rotating). In some embodiments, the solvent evaporation unit is integrated with the receptacle. In other embodiments, the receptacle may be automatically transferred to the solvent evaporation unit. The solvent evaporation unit may be controllable by the controller when removal of a volatile solvent is required in the methods described herein.

The controller may be adapted to carry out the following steps:
contacting the organic phase comprising the dissolved or extracted foodstuff with an excess of at least 30% hydrogen peroxide from the second well and from the third well the compound of the general formula (I):

R¹—COOH (I);
in which R¹ represents H or the general partial formula (II)

(R²)(R³)C(R⁴)— (II)
wherein R², R³ and R⁴, independently of one another, are H, F, Cl, OH or methyl, preferably in the presence of a catalytic amount of a mineral acid at a temperature of from about 20° C. to about 70° C. for about 5 min to about 2 h;
partitioning the solution by adding water from the fourth well to provide an aqueous phase and a reacted organic phase, and
operating the separator to separate the reacted organic phase for analysis of mineral oil contamination in the foodstuff; and
optionally analysing the sample in the analyser.

In some embodiments, the controller may be adapted to also carry out the following step prior to the epoxidation step:
(i) in the receptacle, contacting and dissolving, or extracting, the foodstuff sample with an excess of a solution of hydrocarbon solvent from the first well, and optionally an alcohol from the second well, the hydrocarbon solvent and optional alcohol provided at a temperature of from about 20° C. to the boiling point of the solvent mixture;
(ii) cooling the solution in the receptacle, and if the alcohol was used in the previous step, initiating phase separation by addition of water or a mixture of water and alcohol; from the fourth well;

The controller may preferably be adapted to carry out the above steps (i) and (ii) when the sample is an edible oil. For other foodstuffs, it may be preferred to carry out steps (i) and (ii) manually before providing the sample to the at least partially automated system.

In some embodiments, the controller is also adapted to operate the solvent evaporation unit to substantially remove a hydrocarbon solvent from a dissolved or extracted foodstuff in the receptacle, and preferably adding an amount of a halogenated aliphatic or aromatic hydrocarbon from the sixth well.

In some embodiments, the controller is adapted to add an amount of a halogenated aliphatic or aromatic hydrocarbon from the sixth well to the receptacle prior to the addition of the hydrogen peroxide and/or compound of the general formula (I).

In some embodiments, the controller is also adapted to operate the solvent evaporation unit to substantially remove a halogenated aliphatic or aromatic hydrocarbon from the receptacle, and adding a hydrocarbon solvent from the first well.

In some embodiments, the controller is also adapted to operate the solvent evaporation unit to substantially evaporate the sample to dryness, and optionally adding a hydrocarbon solvent from the first well.

In embodiments, the analyser may be any device suitable for detection of MOSH/MOAH in the hydrocarbon phase. Accordingly, in embodiments, the analyser may comprise an LC-GC-FID, or an HPLC coupled with a GC-MS or GCXGC-MS.

The controller may be adapted to control the supply of solvent and/or reagent from the optional fifth and/or sixth and/or seventh wells to the receptacle.

In some embodiments, the system may comprise a column to further purify the hydrocarbon phase taken from the receptacle before reaching the analyser. The column may be any suitable column, such as alumina, silica or size-exclusion gel. In preferred embodiments, the system does not comprise a silica gel column (other than a column included in an analyser that utilises a silica chromatography component).

The third aspect provides use of a system of the second aspect to conduct a method described herein.

### STATEMENTS OF EMBODIMENTS

Various embodiments of the aspects of this disclosure are set out below.

1. A method for preparing a sample for analysing mineral oil contamination in a foodstuff, the method comprising:
(i) contacting and dissolving, or extracting, the foodstuff with an excess of a (1:x, x:0-10, v/v) solution comprising n-hexane and optionally ethanol at a temperature of from about 20° C. to the boiling point of the solvent mixture;
(ii) cooling the solution, and if ethanol was used in the previous step initiating phase separation by addition of water or a mixture of water and ethanol (1:x, x:0-10, v/v);
(iii) contacting the organic phase produced in the previous step with an excess of at least 30% hydrogen peroxide and a compound of the general formula (I)

R¹—COOH (I);
in which R¹ represents H or the general partial formula (II)

(R²)(R³)C(R⁴)— (II),
wherein R², R³ and R⁴, independently of one another, represent H, F, Cl, OH or methyl, in the presence of a catalytic amount of a mineral acid at a temperature of about 20° C. to about 70° C. for about 5 min to about 2 h
(iv) partitioning the solution by adding an excess of water to a provide an aqueous phase and an n-hexane phase, and
(v) separating the n-hexane phase for analysis of mineral oil contamination in the foodstuff; and
(vi) optionally analysing the sample.

2. The method of embodiment 1 characterized in that prior to the epoxidation step (iii) the organic phase is evaporated and an amount of halogenated aliphatic or aromatic hydrocarbon is added, wherein the amount is an amount necessary for to dissolve the sample.

3. The method of embodiment 1 characterized in that the epoxidation step (iii) is conducted in the presence of a halogenated aliphatic or aromatic hydrocarbon and the method further comprises after step (iii) evaporating the organic phase and exchanging the solvent for n-hexane.

4. A method for preparing a sample for analysing mineral oil contamination in a foodstuff, wherein the method is at least partially automated, the method comprising:
(i) contacting and dissolving, or extracting, the foodstuff with an excess of a (1:x, x:0-10, v/v) solution comprising n-hexane and optionally ethanol at a temperature of from about 20° C. to the boiling point of the solvent mixture;
(ii) cooling the solution, and if ethanol was used in the previous step initiating phase separation by addition of water or a mixture of water and ethanol (1:x, x:0-10, v/v);
(iii) contacting the organic phase produced in the previous step with an excess of at least 30% hydrogen peroxide and a compound of the general formula (I)

R¹—COOH (I);
in which R¹ represents H or the general partial formula (II)

(R²)(R³)C(R⁴)— (II),
wherein R², R³ and R⁴, independently of one another, represent H, F, Cl, OH or methyl, in the presence of a catalytic amount of a mineral acid at a temperature of about 20° C. to about 70° C. for about 5 min to about 2 h
(iv) partitioning the solution by adding an excess of water to a provide an aqueous phase and an n-hexane phase, and
(v) separating the n-hexane phase for analysis of mineral oil contamination in the foodstuff; and
(vi) optionally analysing the sample.

5. The method of embodiment 1 characterized in that prior to the epoxidation step (iii) the organic phase is evaporated and an amount of halogenated aliphatic or aromatic hydrocarbon is added, wherein the amount is an amount necessary for to dissolve the sample; or.
characterized in that the epoxidation step (iii) is conducted in the presence of a halogenated aliphatic or aromatic hydrocarbon and the method further comprises after step (iii) evaporating the organic phase and exchanging the solvent for n-hexane.

6. The method of embodiment 4 or 5 characterized in that at least 2 steps, or at least 3 steps, or at least 4 steps, or at least 5 steps, or at least 6 steps of the method are automated and run without operator involvement.

7. The method of embodiment 4 or 5 characterized in that the method is fully automated.

8. The method according to any one of the preceding embodiments, characterized in that, the foodstuff comprises alkaline hydrolyzable constituents in the organic phase and following step (ii) and prior to step iii the alkaline hydrolyzable constituents are saponified by:
contacting the dissolved or extracted foodstuff with an excess of a concentrated aqueous alkali metal hydroxide solution or other strong base above about 20° C. to about 70° C., and
extracting the non-saponified constituents by adding n-hexane; and
optionally repeating the extraction with n-hexane one or more times.

9. The process according to any one of the embodiments 1 to 7, characterized in that the foodstuff comprises alkaline hydrolyzable constituents in the organic phase and following step (ii) and prior to step iii the alkaline hydrolyzable constituents are saponified by:
contacting the dissolved or extracted foodstuff with an excess of a concentrated aqueous alkali metal hydroxide solution or other strong base above about 20° C. to about 70° C., and
extracting the non-saponified constituents by adding n-hexane; and
optionally repeating the extraction with n-hexane one or more times.

10. The method according to any one of the embodiments 1 to 7 characterized in that, the food stuff is a dried foodstuff or is a foodstuff present in aqueous phase and, prior to step (i) the mineral oil impurities are extracted from the foodstuff by:
contacting the foodstuff with an excess of an organic water-insoluble solvent; and
separating the solvent comprising extracted mineral oil impurities from the foodstuff using suitable means.

11. The process according to any one of the embodiments 1 to 10, characterized in that the dissolution/extraction of the mineral oil components in the food is carried out with an excess of a (1:x, x:0-10, v/v) solution of n-hexane and 96 vol % or absolute ethanol at a temperature from about 20° C. to the boiling point of the solvent mixture.

12. The process according to any one of the embodiments 8 to 11, characterized in that the saponification of the organic phase is carried out with an excess of a concentrated alkali metal hydroxide solution in the form of sodium hydroxide solution and/or potassium hydroxide solution at from about 20° C. to about 70° C.

13. The process according to any one of the embodiments 1 to 12, characterized in that the halogenated hydrocarbon chloroform is added to the organic phase before the epoxidation, step (iii), is started.

14. The process according to any one of the embodiments 1 to 13, characterized in that the epoxidation, step (iii), comprises
contacting the organic phase with a mixture of 50% hydrogen peroxide and concentrated formic acid and/or acetic acid in the presence of a catalytic amount of concentrated phosphoric acid and/or sulfuric acid at a temperature of from about 20° C. to about 70° C. for about 10 min to about 60 min.

15. A method according to any one of the embodiments 1 to 14, characterized in that the foodstuff is a substance or product intended to be or reasonably expected to be ingested by humans in a processed, partially processed or unprocessed state and in particular selected from natural fats, protein-containing foods, carbohydrate-rich foods, alcohol-containing foods, alkaloid-containing foods, vegetables and vegetable products, fruits and fruit products, spices or herbs, drinking water, soft drinks, functional foods, food supplements, dietary foods, novel foods, vitamins, minerals, enzymes, lipids, amino acids, additives or mixtures of the aforementioned substances and/or products of the food classes.

16. The method according to any one of the embodiments 1 to 12 characterized in that the natural fat is a fruit pulp fat or seed fat or an animal fat, or a mixture of the aforementioned fats, preferably an edible oil or fat.

17. The method according to any one of the embodiments 1 to 12, characterized in that the fat-containing food is milk or a milk product, chocolate, chocolate products, cocoa, margarine products, mixed fat products, infant formula and follow-on formula, or a mixture of the aforementioned products.

18. A method for preparing a sample for analysing mineral oil contamination in a foodstuff, the method comprising:
(i) contacting and dissolving, or extracting, the foodstuff with an excess of a (1:x, x:0-10, v/v) solution comprising n-hexane and optionally ethanol at a temperature of from about 20° C. to the boiling point of the solvent mixture;
(ii) cooling the solution, and if ethanol was used in the previous step initiating phase separation by addition of water or a mixture of water and ethanol (1:x, x:0-10, v/v);
(iii) contacting the organic phase produced in the previous step with an excess of at least 30% hydrogen peroxide and a compound of the general formula (I)

R¹—COOH (I)
in which R¹ represents H or the partial general formula (II)

(R²)(R³)C(R⁴)— (II)
wherein R², R³ and R⁴, independently of one another, represent H, F, Cl, OH or methyl, in the presence of a catalytic amount of a mineral acid at a temperature of about 20° C. to about 70° C. for about 5 min to about 2 h
(iv) partitioning the solution by adding an excess of water to a provide an aqueous phase and an n-hexane phase, and
(v) separating the n-hexane phase for analysis of mineral oil contamination in the foodstuff; and
(vi) optionally analysing the sample.

19. A method for preparing a sample for analysing mineral oil contamination in a foodstuff, wherein the method is at least partially automated, the method comprising:
(i) contacting and dissolving, or extracting, the foodstuff with an excess of a (1:x, x:0-10, v/v) solution comprising n-hexane and optionally ethanol at a temperature of from about 20° C. to the boiling point of the solvent mixture;
(ii) cooling the solution, and if ethanol was used in the previous step initiating phase separation by addition of water or a mixture of water and ethanol (1:x, x:0-10, v/v);
(iii) contacting the organic phase produced in the previous step with an excess of at least 30% hydrogen peroxide and a compound of the general formula (I)

R¹—COOH (I)
in which R¹ represents H or the partial general formula (II)

(R²)(R³)C(R⁴)— (II)
wherein R², R³ and R⁴, independently of one another, represent H, F, Cl, OH or methyl, in the presence of a catalytic amount of a mineral acid at a temperature of about 20° C. to about 70° C. for about 5 min to about 2 h
(iv) partitioning the solution by adding an excess of water to a provide an aqueous phase and an n-hexane phase, and
(v) separating the n-hexane phase for analysis of mineral oil contamination in the foodstuff and,
(vi) optionally analysing the sample.

20. The method of embodiment 18 or 19 wherein prior to the epoxidation step (iii) the organic phase is evaporated and an amount of halogenated aliphatic or aromatic hydrocarbon is added, wherein the amount is an amount necessary for to dissolve the sample.

21. The method of embodiment 18 or 19 wherein the epoxidation step (iii) is conducted in the presence of a halogenated aliphatic or aromatic hydrocarbon and the method further comprises after step (iii) evaporating the organic phase and exchanging the solvent for n-hexane.

21. The method of any one of embodiments 19 to 21 wherein the method is fully automated.

22. The method of any one of embodiments 19 to 21 wherein at least 2 steps, or at least 3 steps, or at least 4 steps, or at least 5 steps, or at least 6 steps of the method run without operator involvement.

23. The method of any one of embodiments 19 to 21 wherein the method is fully automated.

24. The method according to any one of embodiments 18 to 23, wherein, the foodstuff comprises alkaline hydrolyzable constituents in the organic phase and following step (li) and prior to step iii the alkaline hydrolyzable constituents are saponified by:
contacting the dissolved or extracted foodstuff with an excess of a concentrated aqueous alkali metal hydroxide solution or other strong base above about 20° C. to about 70° C., and
extracting the non-saponified constituents by adding n-hexane; and
optionally repeating the extraction with n-hexane one or more times.

25. The process according to any one of the embodiments 18 to 23, wherein the foodstuff comprises alkaline hydrolyzable constituents in the organic phase and following step (ii) and prior to step (iii) the alkaline hydrolyzable constituents are saponified by:
contacting the dissolved or extracted foodstuff with an excess of a concentrated aqueous alkali metal hydroxide solution or other strong base above about 20° C. to about 70° C., and
extracting the non-saponified constituents by adding n-hexane; and
optionally repeating the extraction with n-hexane one or more times.

26. The method according to any one of the embodiments 18 to 23, wherein the food stuff is a dried foodstuff or is a foodstuff present in aqueous phase and, prior to step (i) the mineral oil impurities are extracted from the foodstuff by
contacting the foodstuff with an excess of an organic water-insoluble solvent; and
separating the solvent comprising extracted mineral oil impurities from the foodstuff using suitable means.

27. The process according to any one of the embodiments 18 to 26, wherein the dissolution/extraction of the mineral oil components in the food is carried out with an excess of a (1:x, x:0-10, v/v) solution of n-hexane and 96 vol % or absolute ethanol at a temperature from about 20° C. to the boiling point of the solvent mixture.

28. The method according to any one of the embodiments 18 to 27, wherein the saponification of the organic phase is carried out with an excess of a concentrated alkali metal hydroxide solution in the form of sodium hydroxide solution and/or potassium hydroxide solution at from about 20° C. to about 70° C.

29. The process according to any one of the embodiments 18 to 28, wherein the halogenated hydrocarbon chloroform is added to the organic phase before the epoxidation, step (iii), is started.

30. The process according to any one of the embodiments 18 to 29, wherein the epoxidation, step (iii), comprises
contacting the organic phase with a mixture of 50% hydrogen peroxide and concentrated formic acid and/or acetic acid in the presence of a catalytic amount of concentrated phosphoric acid and/or sulfuric acid at a temperature of from about 20° C. to about 70° C. for about 10 min to about 60 min.

31. A method according to any one of the embodiments 1 to 30, wherein the foodstuff is a substance or product intended to be or reasonably expected to be ingested by humans in a processed, partially processed or unprocessed state and in particular selected from natural fats, protein-containing foods, carbohydrate-rich foods, alcohol-containing foods, alkaloid-containing foods, vegetables and vegetable products, fruits and fruit products, spices or herbs, drinking water, soft drinks, functional foods, food supplements, dietary foods, novel foods, vitamins, minerals, enzymes, lipids, amino acids, additives or mixtures of the aforementioned substances and/or products of the food classes.

32. The method according to embodiment 31 wherein the natural fat is a fruit pulp fat or seed fat or an animal fat, or a mixture of the aforementioned fats, preferably an edible oil or fat.

33. The method according to any one of embodiments 18 to 31, wherein the fat-containing foodstuff is milk or a milk product, chocolate, chocolate products, cocoa, margarine products, mixed fat products, infant formula and follow-on formula, or a mixture of the aforementioned products.

34. The method according to any one of embodiments 18 to 31 wherein the fat containing foodstuff comprises palm oil.

35. Method for the (partially) automated analysis of mineral oil contamination in foodstuffs with the following steps in sample preparation:
Contacting and dissolving or extracting the food with an excess of a (1:x, x:0-10, v/v) solution of n-hexane and ethanol at a temperature between 20° C. and the boiling point of the solvent mixture.
after cooling and if ethanol was used in the previous step, initiation of phase separation by addition of water or a mixture of water and ethanol (1:x, x:0-10, v/v)
contacting the organic phase with an excess of at least 30% hydrogen peroxide and a compound of the general formula (I) R¹—COOH (I) in which R¹ represents H or the general formula (II) (R²)(R³)C(R⁴)— (II) where R², R³ and R⁴, independently of one another, represent H, F, Cl, OH or methyl, in the presence of a small amount of a mineral acid at a temperature of above 20° C. to 70° C. for 5 min to 2 h with epoxidation
separation of the reagents by adding an excess of water; and
use of the n-hexane extract for the following analysis
   characterized in that the epoxidation step is preceded by evaporation of the organic phase and addition of an amount of halogenated aliphatic or aromatic hydrocarbon necessary for solution, or in that the epoxidation step is followed by evaporation of the organic phase and exchange of the solvent for n-hexane in the presence of a halogenated aliphatic or aromatic hydrocarbon.

36. The process according to embodiment 35, characterized in that, in the case of foodstuffs with alkaline hydrolyzable constituents in the organic phase, saponification is carried out after the first step by intimate contact with an excess of a concentrated aqueous alkali metal hydroxide solution or other strong base above 20° C. to 70° C., the subsequent extraction being carried out by adding n-hexane at least once.

37. The process according to embodiment 35, characterized in that, in the case of foodstuffs with acidic hydrolyzable constituents in the organic phase, hydrolysis is carried out after the first step by intimate contact with an excess of a concentrated aqueous hydrochloric acid solution or other strong acid above 20° C. to 70° C., the subsequent extraction being carried out by adding n-hexane at least once.

38. The method according to embodiment 35, characterized in that, in the case of dried foodstuffs or foodstuffs present in aqueous phase, prior to the first step of sample preparation, extraction of the mineral oil impurities from the foodstuff is carried out in a manner known per se with an excess of an organic water-insoluble solvent in a manner known per se.

39. The process according to embodiment 35, characterized in that the dissolution/extraction of the mineral oil components in the food is carried out with an excess of a (1:x, x:0-10, v/v) solution of n-hexane and 96 vol % or absolute ethanol at a temperature between 20° C. and the boiling point of the solvent mixture.

40. The process according to embodiment 36, characterized in that the saponification of the organic phase is carried out with an excess of a concentrated alkali metal hydroxide solution in the form of sodium hydroxide solution and/or potassium hydroxide solution at 20° C. to 70° C.

41. The process according to embodiment 35, characterized in that the halogenated hydrocarbon chloroform is added to the organic phase before the epoxidation is started.

42. The process according to embodiment 35, characterized in that the epoxidation is carried out by contacting the organic phase with a mixture of 50% hydrogen peroxide and concentrated formic acid and/or acetic acid in the presence of a small amount of concentrated phosphoric acid and/or sulfuric acid at a temperature of above 20° C. to 70° C. for 10 min to 60 min.

43. A method according to any one of embodiments 35 to 42, characterized in that the food is substances or products intended to be or reasonably expected to be ingested by humans in a processed, partially processed or unprocessed state and in particular selected from natural fats, protein-containing foods, carbohydrate-rich foods, alcohol-containing foods, alkaloid-containing foods, vegetables and vegetable products, fruits and fruit products, spices or herbs, drinking water, soft drinks, functional foods, food supplements, dietary foods, novel foods, vitamins, minerals, enzymes, lipids, amino acids, additives or mixtures of the aforementioned substances and/or products of the food classes.

44. The method according to embodiments 43, characterized in that the natural fat is a fruit pulp fat or seed fat or an animal fat, or a mixture of the aforementioned fats, preferably an edible oil or fat.

45. The method according to embodiments 43, characterized in that the fat-containing food is milk or a milk product, chocolate, chocolate products, cocoa, margarine products, mixed fat products, infant formula and follow-on formula, or a mixture of the aforementioned products.

### EXAMPLES

The present invention will now be described by way of non-limiting example(s). It will be understood to persons skilled in the art of the invention that many modifications may be made without departing from the spirit and scope of the invention.

### Example 1

As an example, one gram of a palm olein (iodine value 62-64) was weighed into a 20 mL screw-cap glass vial with a blank septum and dissolved in 10 mL of n-hexane/ethanol (1:1, v/v). 10 μL of an internal standard was added (Restek GmbH, #31070, w=300 ng/μL). Subsequently, 2 mL of an aqueous potassium hydroxide solution (50%, w/v) was added. The sample was saponified by an autosampler for 20 min at 65° C. as well as 750 rpm. After addition of 4 mL of water, the sample was automatically centrifuged (2 min, 3500 rpm). The obtained upper phase was transferred through a syringe into a new screw-cap glass vial. Another 5 mL of n-hexane was added and the sample was shaken. After subsequent centrifugation (2 min, 3500 rpm). the upper phases were combined. At a vacuum of 200 mbar and with shaking at 250 rpm (65° C.), the organic phase was reduced to approximately 1 mL in volume. This was followed by the addition of 2 mL of chloroform as well as 70 μL of H3PO4 (Sigma Aldrich, 85 wt. %, aq), 700 μL of formic acid (Sigma Aldrich, 98-100%), and 700 μL of H2O2 (Bernd kraft, 50%, aq). With vigorous shaking of 750 rpm, the extract was epoxidized at 65° C. for 30 min. The reaction was stopped by adding 7.5 mL of water. To better transfer the organic lower phase, 2.5 mL of n-hexane was added for phase inversion. Thus, the upper phase could be transferred to a new screw-cap glass vial.

The organic phase was carefully concentrated under vacuum (200 mbar, 250 rpm, 65° C.) after addition of a keeper (50 μL bis(2-ethylhexal)-sebacate, Sigma Aldrich, >90%) and made up with 1.5 mL n-hexane. To remove the last suspended solids, the solution was centrifuged again (2 min, 3500 rpm).

Up to 400 μL of the extract thus obtained could be injected into an LC-GC-FID system. The hexane extract injected into the HPLC column was separated on a silica gel phase by means of normal phase gradient (hexane/DCM) into the substance classes MOSH and MOAH as well as polar components. Here, the HPLC column completely takes over the task of silica gel clean-up from previous sample instructions. The fraction sections for MOSH and MOAH each comprised a volume of 600 μL. This was constricted by a large volume on-column transfer interface using partial simultaneous solvent evaporation and early vapor exit. The two GC-compatible fractions obtained were separated on suitable capillary columns according to their boiling points and detected via flame ionization.

At the same time, the flow direction of the HPLC column was reversed (backflush) to efficiently remove polar residues from the column. The total process until the next sample was ready for injection took about 35 min.

If purification via alumina is necessary for the MOSH fraction, the corresponding fraction is passed from the silica gel column through an Al2O3 column before being transferred to the gas chromatograph. In this way, no manual ALOX-clean-up is required.

### Example 2

Example 1 was repeated, but instead of a refined palm olein, 1 g of an oil mixture of extra native olive oil and refined sunflower oil (1:1, v/v) was reprocessed. The workup can be 100% analogous, but allows for simplification if desired.

If an edible oil contains only olefins with moderate or high electron density, as is often the case with native edible oils, the solvent change to halogenated hydrocarbons can be omitted, i.e. epoxidation can be carried out directly from the (saponified) hexane phase. This procedure ensures an acceleration of the test method presented here.

### Example 3—Sample Preparation According to DGF

The fat according to Example 1 was subjected to sample preparation according to DGF as follows:

3 g of sample was weighed into a 40 mL screw-capped centrifuge tube. 30 mL of a mixture of n-hexane and ethanol (1:1, v/v) and 10 μL ISTD (w=300 ng/μL) were added and shaken. To a sample vial, 10 mL was transferred and 3 mL of potassium hydroxide solution (33 g/100 g water) was added. The solution was saponified for 30 min at 60° C. in a water bath with shaking. Subsequently, 5 mL each of n-hexane and 5 mL of the mixture of ethanol and water (1:1, v/v) were added, the mixture was shaken again, and after phase separation the lower phase was discarded.

Since palm olein contains only small amounts of biogenic n-alkanes, ALOX purification was not necessary. The upper phase from the saponification was transferred directly to a clean-up column (3 g silica gel+1 g Na₂SO₄) and the hydrocarbons (MOSH+MOSH) were eluted from the column with 15 mL solvent mixture of n-hexane and DCM (7:3, v/v).

After addition of 2 drops of bis(2-ethylhexyl)maleate, the mixture was concentrated at 40° C. in vacuo while maintaining all internal standards and made up to a volume of 1 mL with n-hexane.

To the extract thus obtained, 1 mL of ethanolic CPBA solution (100 mg/mL) was added and the sample was placed on a shaker for 20 min at 40° C. at, for example, 500 rpm. Then, 500 μL ethanol and 2 mL solution (Na₂S₂O₃, 5 g/100 mL+NaHCO₃, 5 g/100 mL in water) were used to react off the excess CPBA. The sample vial was shaken for about 1 min at about 750 rpm to react off the excess CPBA. The upper hexane phase was transferred to a fresh sample vial and dried with a spatula tip of sodium sulfate. The dried solution was now injected into an LC-GC-FID-system at an injection volume of 60-90 μL. To allow reproducible evaluation at low levels near the limit of quantitation of 1 mg/kg, the solvent was adjusted to a more appropriate volume of approximately 300 μL at 40° C. while maintaining the internal standards.

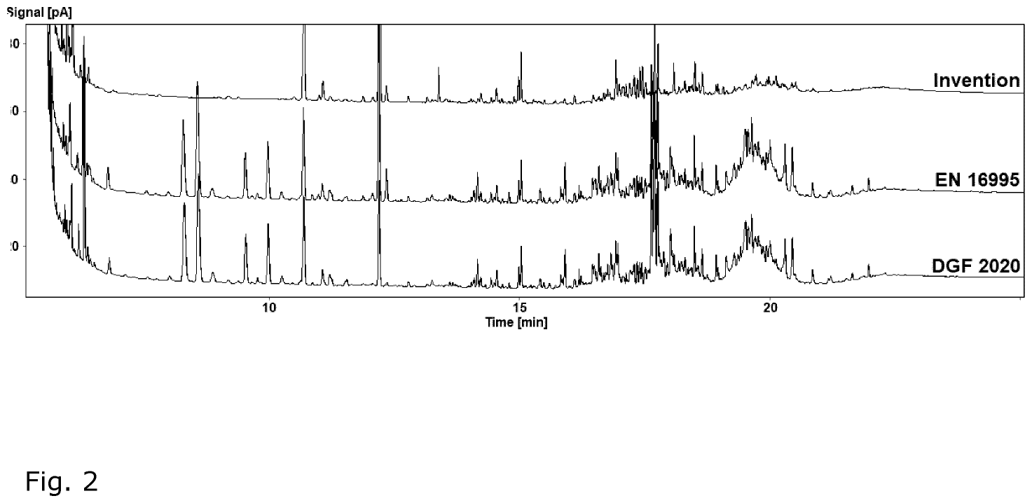

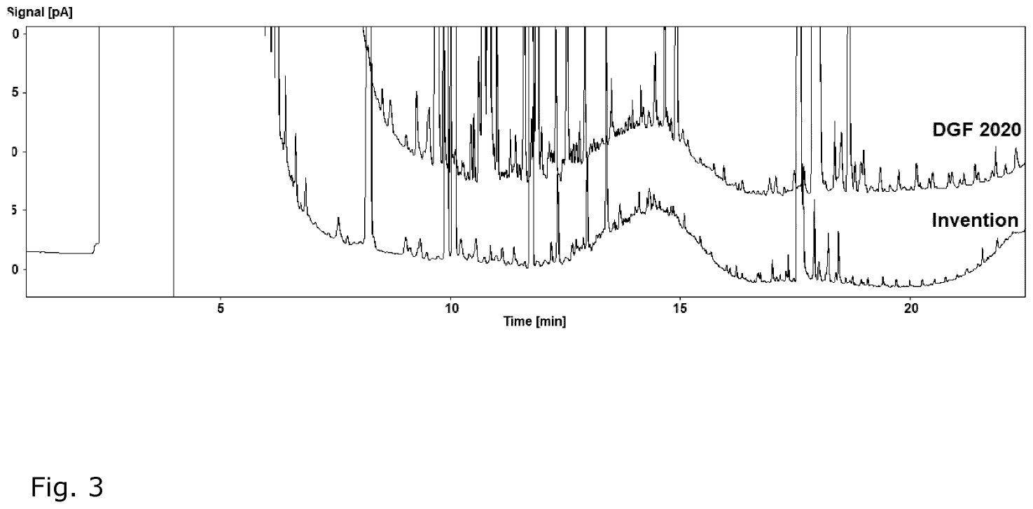

FIG. 2 shows an LC-GC-FID chromatogram of the MOAH fraction of a palm olein after sample preparation according to the invention, example 1, (uppermost chromatogram) compared with example 3 [EN 16995, DGF-2020]. In the right panel: time less than 20 min, chromatograms 2 and 3 below show significantly unresolved signal peaks that could be misinterpreted as MOAH, whereas these peaks are significantly reduced in the top chromatogram [according to the invention]. (All three methods were performed for comparison such that the amount of sample injected was identical). Because unresolved signal humps typically represent mineral oil contamination, a significantly lower false positive identification rate can be expected when using the test method presented here. Coupled with the simplified sample preparation and increased injection volume (400 μL vs. 60-90 μL), the identification confidence near the limit of quantitation also increases.

FIG. 3 shows an LC-GC chromatogram of the MOAH fraction of a 1:1 mixture of olive and sunflower oil, after sample preparation to which a mineral oil has been added to simulate contamination. The upper chromatogram shows a preparation according to DGF 2020, comparative example, and the lower curve shows the method according to the invention, example 2. The MOAH content is 2.5 mg/kg. The unresolved signal hump (or unresolved signal peak) between 12 and 16 min corresponds to this amount. Also for this “simple” oils, the new method exhibits improved chromatography. The entire chromatogram, the unresolved signal hump (or unresolved signal peak) in particular, shows fewer interfering individual peaks or peak clusters. (For the comparison, both methods were performed in such a way that the injected sample amount is identical).

### Example 4

### Materials and Methods

### Samples

Extra virgin olive oil was obtained at the local supermarket and used for method development and validation. Additionally, edible oil samples from a collaborative trial performed in 2020 organized by the DGF during validation of standard method C-VI 22 (20) were available. They consisted of cocoa butter, sunflower oil, spiked rapeseed oil, spiked olive oil, spiked sunflower oil, and spiked palm oil. EIE (enzymatic interesterification) and RBD palm oils were private gifts.

### Chemicals and Solutions

n-Hexane was from Th. Geyer GmbH & Co. KG (CHEMSOLUTE, Renningen, Germany). The internal standard (ISTD) for MOH quantitation (Cat. No. 31070—n-undecane, n-tridecane, bicyclohexyl, α-cholestane, n-pentylbenzene, 1-methylnaphthalene, 2-methylnaphthalene, 1,3,5-tri-tert-butylbenzene, perylene), retention time standard (Cat. No. 31076), and EPA-PAH standard (Cat. No. 31011) were supplied by Restek (Bellefonte, PA, USA). Industrial gear oil (Omala S2 GX 68) and naphthenic process oil (Gravex 913) for spiking experiments were obtained from Shell Deutschland GmbH (Hamburg, Germany). Bulk aluminum oxide (90 active basic, 0.063-0.200 mm), bis(2-ethylhexyl) sebacate (for synthesis), chloroform (HPLC Plus, ≥99.9%), dichloromethane (Suprasolv), ethanol (Suprasolv), β-carotene (≥93% UV), 3,5-cholestadiene (≥93% HPLC), dibenzofurane (DBF, 98%), dibenzothiophene (DBT, 98%), formic acid (EMPROVE ESSENTIAL, 98-100%), meta-chloroperoxybenzoic acid (≥77%), phosphoric acid (85 wt. % in H2O), iso-octane (Suprasolv), potassium hydroxide (EMSURE, 85%), sodium metabisulfite (ReagentPlus, ≥99%), squalene (98%), 1,13-tetradecadiene (90%), and 1-octadecene (analytical standard) were from Merck (Steinheim, Germany). Sodium sulfate was from Fluka (Buchs, Switzerland). Hydrogen peroxide (50%, w/v) was obtained from Berndt Kraft GmbH (Duisburg, Germany). Water was supplied by a Milli-Q water purification system (Merck, Darmstadt, Germany).

### 3A Epoxidation of Model Substances for Kinetic Studies

To a 1 mL mixture of used model substances in n-hexane or chloroform (20 μg/mL, w/v), the autosampler added 200 μL of formic acid (acidified with 10% H₃PO₄, v/v) and 200 μL of aqueous H₂O₂ to the sample. The vial was placed into an agitator and was shaken at a speed of 750 rpm (revolutions per minute) for 5, 10, 15, 20, 25, and 30 min at 65° C. During the reaction, considerable amounts of oxygen and carbon dioxide were formed by side reactions building up a pressure in the autosampler vial. The vial was therefore tightly closed.

Afterward, 2.5 mL water were added to stop the reaction and induce phase separation. The vial was shaken at 750 rpm for 1 min. Thereafter, the vial was centrifuged for 1 min at 2000 rpm to get a clear organic phase. Five hundred microliters of the n-hexanic upper phase were transferred into a 2-mL autosampler vial prefilled with a spatula tip of sodium sulfate. The dried organic phase was subjected to LC-GC-FID.

### Sample Preparation Workflow

### Saponification

One gram of fat or oil was weighed into a 20-mL autosampler vial. Five microliters of ISTD (300 μg/mL, w/v) were added as well as 10 mL of n-hexane/ethanol (1:1, v/v). After addition of 2 mL of aqueous KOH solution (1 g/L, w/v), the vial was tightly closed. Saponification was performed at 65° C. for 20 min with steady shaking at 750 rpm. Thereafter, 4 mL of water were added, and the vial intensively shaken for 1 min at 750 rpm. After centrifugation for 1 min at 3000 rpm, the clear upper n-hexanic phase was transferred into a fresh 20-mL autosampler vial. A second extraction was performed by adding 5 mL of fresh n-hexane to the saponified sample, shaking and centrifugation. Both hexane phases were combined resulting in a final volume of approximately 8.5 mL.

### Epoxidation

Fifty microliters of bis(2-ethylhexyl) sebacate were added to the n-hexanic extract of a saponified sample and the solvent was evaporated by vacuum (65° C., 200 mbar) to a volume of approximately 1 mL. Thereafter, the autosampler added 2 mL of CHCl₃, 400 μL of formic acid (acidified with 10% H₃PO₄, v/v) and 400 μL of aqueous H₂O₂ to the sample. The vial was placed into an agitator and was shaken at a speed of 750 rpm for 20 min at 65° C.

Afterward, a mixture of 3.5 mL of n-hexane, 1 mL of ethanol and 7 mL of water were added to stop the reaction and induce phase separation. The added n-hexane and ethanol were used for a phase layer inversion allowing to aspirate the organic layer from the top of the vial. The vial was shaken at 750 rpm for 1 min. Thereafter, the vial was centrifuged for 1 min at 4000 rpm to get a clear organic phase.

Six millilitres of the upper organic phase were transferred into a 10-mL autosampler vial prefilled with a spatula tip of sodium sulfate and gently evaporated to dryness (65° C., 200 mbar). Finally, the residue was dissolved in 1.5 mL n-hexane, shaken at 750 rpm for 1 min, and centrifuged at 4000 rpm for 5 min. The clear organic phase was subjected to LC-GC-FID.

Excess H₂O₂ in the aqueous phase of the 20-mL autosampler vial was destroyed by slow addition of 2 mL of an aqueous sodium metabisulfite solution (300 g/L).

### LC-GC-FID Method

LC-GC-FID experiments were performed on a CHRONECT Workstation MOSH/MOAH from Axel Semrau (Sprockhövel, Germany). It consisted of a 1260 Infinity II HPLC system (binary and quaternary pump and variable wavelength detector by Agilent Technologies, Waldbronn, Germany), Agilent 7890B gas chromatograph with two flame ionization detectors and the CHRONECT Robotics platform based on a PAL3 autosampler (CTC Analytics AG, Zwingen, Switzerland).

Four rotatory switching valves (VICI AG International, Schenkon, Switzerland) were used to guide the HPLC eluent from the HPLC into the GC. The gas chromatograph was equipped with two on-column interfaces (Y-interface) and solvent vapor exits.

Typically, 400 μL of the prepared sample (corresponding to 267 mg of edible oil or fat) were injected onto an Allure Si HPLC column (250 mm×2.1 mm, 5 μm, 60 Å, Restek, Bellefonte, PA, USA) without additional column temperature control. The mobile phase consisted of n-hexane and dichloromethane. Starting at 100% n-hexane with 300 μL/min, the mobile phase was changed to 70% n-hexane after injection. It was held until 7.5 min. MOSH was eluted from 2.0-4.0 min (600 μL) through the manually packed aluminium oxide HPLC column (125 mm×2.1 mm, sorbent was activated for 16 h at 500° C. prior to use) for removal of biogenic n-alkanes. After elution of the MOAH fraction (4.1-6.1 min, 600 μL), the column was back flushed with dichloromethane at 500 μL/min for 9 min. Afterward, the column was reconditioned with n-hexane at 500 μL/min for 15 min. At the same time, the aluminium oxide HPLC column was back flushed with iso-octane at 1 mL/min for 10 min prior to reconditioning with n-hexane at 1 mL/min for 10 min. The MOAH elution window was verified by UV detection at 230 nm. 1,3,5-tri-tert-butylbenzene (TBB) and perylene (Per) marked the starting- and endpoint of this window.

LC-GC transfer occurred by the retention gap technique and partially concurrent solvent evaporation (PCSE). An uncoated, deactivated pre-column (Deactivated stainless steel, 10 m×0.53 mm, Axel Semrau, Sprockhövel, Germany) was followed by a steel T-piece union (modified butt-to-butt connector, Trajan Scientific and Medical, Ringwood, Australia) connecting to the solvent vapor exit and a separation column coated with a 100% dimethyl polysiloxane film (MXT-1, Siltek-treated stainless steel, 15 m×0.25 mm×0.25 μm, Restek, Bellefonte, PA, USA).

From HPLC, the MOSH and MOAH fractions were transferred to GC at a carrier gas inlet pressure of 95 and 55 kPa (hydrogen), respectively, and an oven temperature of 60° C. The solvent vapor exit was opened 0.5 min prior to elution of each fraction and was closed 0.4 min after the fraction was transferred. Recoveries of undecane and n-pentylbenzene were quantitative under these conditions. At the same time, the carrier gas inlet pressure was set to 150 kPa and maintained throughout the whole analysis. The oven temperature was programmed at 20° C./min from 60° C. (8 min) to 370° C. (6.5 min, total run time 30.00 min). The FID base temperature was set to 380° C. The gas flows for air, hydrogen, and nitrogen were set to 300, 30, and 25 mL/min, respectively.

Data processing was performed with Clarity 8.5 (DataApex, Prague, Czech Republic). Quantitation was based on bicyclohexyl (cyclohexyl cyclohexane, Cycy) for MOSH as well as TBB for MOAH used as ISTDs. The MOxH content was calculated following the equation:

$$C = \frac{( {A}_{M O x H , Total} - {A}_{MOxH , Peaks on Top} ) * {m}_{ISTD}}{{A}_{ISTD} * {m}_{S a m p l e}}$$

with C: Content [mg/kg], AMO×H, Total: Unresolved MO×H hump area, AMO×H, Peaks on Top: Area of sharp peaks on top of the MO×H hump, AISTD: peak area of ISTD, mISTD: mass of ISTD [mg], mSample: mass of test sample [kg].

For identification purposes, the MOSH or MOAH fraction was collected and analyzed by GC-MS (GCMS-QP2020 NX, Shimadzu Europa GmbH, Duisburg, Germany). The ion source and transfer line temperatures were set to 250 and 320° C., respectively. Data acquisition was performed in full-scan mode (50-750 amu) at a rate of 3 spectra/s with EI ionization at 70 eV. Data processing was performed with GCMS solution 4.52.

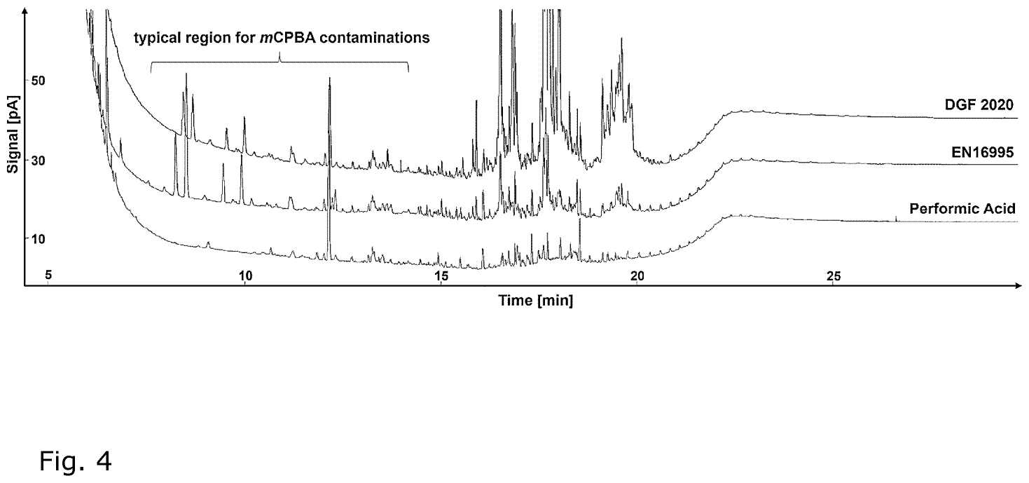

FIG. 4 shows an LC-GC-FID MOAH chromatogram overlay of epoxidized RBD palm oil showing the residual polyunsaturates. Performic acid not only removed more interferences mCPBA, but also showed virtually no contaminations originating from the percid. Epoxidation with mCPBA in ethanol for this sample showed the expected retarding in reaction rate compared to dichloromethane.

One of the problems of the present method for analysing MOSH/MOAH in food oils is the inconsistent quality of mCPBA with a concern regarding contaminants. Through GC-MS investigation it was found that these contaminants originate from the synthesis of the peracid. They are mainly composed of polychlorinated benzenes and biphenyls (PCBs). Although washing of mCPBA with n-hexane already removes a major part, disturbing residues can still be present making additional clean-up mandatory. As pure mCPBA tends to be shock-sensitive, particular care is necessary. For this reason, alternative epoxidation reagents were checked. Since performic and peracetic acid are widely used for epoxidation of edible oils, e.g., to formulate epoxidized soybean oil, these compounds were further analyzed. Both peracids are unstable under ambient conditions, and hence need to be generated in-situ from hydrogen peroxide and formic or acetic acid, respectively. Strong mineral acids, such as H₃PO₄ or H₂SO₄, serve as catalyst to increase the rate of formation.

For that purpose, a challenging matrix like RBD palm oil known to have persistent interferences was treated with both reagents for 30 min at 65° C. As expected performic acid showed higher and faster removal of polyunsaturates than peracetic acid.

Surprisingly, the removal was even higher compared to mCPBA. In FIG. 4, a chromatogram overlay is depicted showing that the MOAH fraction of RBD palm oil after epoxidation with performic acid contained less chromatographic interferences. It is also apparent for this specific sample that epoxidation with mCPBA in ethanol is inferior to dichloromethane. Without wishing to be bound by theory, it is considered that this may be attributed to the polarity of the solvent causing a slowdown in reaction rates. For these reasons and in scope of the present work, performic acid was chosen for further investigations of the nature of epoxidation.

Five model substances resembling typical biogenic olefins were chosen and subjected to epoxidation with performic acid. Where applicable, comparison to traditional epoxidation with mCPBA was made. Epoxidation follows 2nd order kinetics according to the equation:

$$r = - \frac{d [ A ]}{d t} = {k}_{2} [ Peracid ] [ A ]$$

If the peracid is added in excess and its concentration considered constant, the reaction follows pseudo-first order and thus the upper equation can be simplified from which important parameters such as reaction rate constants (k1) and half-life times (t½) can be derived.

$$[ Peracid ] \approx const ⇒ {k}_{1} \approx {k}_{2} [ Peracid ]$$

$$r = - \frac{d [ A ]}{d t} = {k}_{1} [ A ]$$

$$[ A ] = {[ A ]}_{0} {e}^{- {k}_{1} 𝔱} ⇔ ln [ A ] = - {k}_{1} t + {ln [ A ]}_{0}$$

$$⇒ {t}_{1 / 2} = \frac{ln ( 2 )}{{k}_{1}}$$

TABLE 1
Reaction rate constants and half-life times of model olefins subjected to epoxidation

| | | | | | 3,5- | | 1,13- | | | |
|---|---|---|---|---|---|---|---|---|---|---|
| | Squalene | | β-Carotene | | Cholestadiene | | Tetradecadiene | | 1-Octadecene | |
| | k₁ [min⁻¹] | t_{1/2} [min] | k₁ [min⁻¹] | t_{1/2} [min] | k₁ [min⁻¹] | t_{1/2} [min] | k₁ [min⁻¹] | t_{1/2} [min] | k₁ [min⁻¹] | t_{1/2} [min] |
| Performic acid | | | | | | | | | | |
| n-Hexane | 3.89 | 0.2 | 1.25^{a} | 0.6 | 1.09 | 0.6 | 0.051 | 14 | 0.008 | 82 |
| CHCl₃ | high | <0.1 | high | <0.1 | high | <0.1 | 0.58 | 1.2 | 0.28 | 2.5 |
| mCPBA^{b} | | | | | | | | | | |
| EtOH (40° C.) | high | <0.1 | 2.81 | 0.2 | 1.63 | 0.4 | low | ∞ | low | ∞ |
| CH₂Cl₂ (RT) | high | <0.1 | high | <0.1 | high | <0.1 | 0.25^{c} | 2.8 | 0.068^{c} | 10 |

Notes:
^{a}Solubility of β-carotene in n-hexane is low (~0.1 g/L), probably limiting the achievable reaction rate.
^{b}Reaction conditions: 100 mg of mCPBA, 20 min, at room temperature or 40° C. as indicated
^{c}Experiments conducted at 40° C. instead of standard conditions. Higher temperatures were not tried due to the boiling point of CH₂Cl₂.

In Table 1, the results of these experiments are summarized. The epoxidation of squalene and β-carotene is much faster than epoxidation of 3,5-cholestadiene. Without wishing to be bound by theory it is proposed this is due to their conjugated rr-electron systems. The half-life times for epoxidation of 1,13-tetradecadiene and 1-octadecene, were significantly higher. The inventors propose this may be due to the influence of the number and positions of the double bonds on the reaction rate. Additionally, the influence of the solvent's polarity can be seen when comparing the results of mCPBA epoxidation in ethanol and dichloromethane.

While epoxidation performed with mCPBA in dichloromethane showed the highest reaction rates for most compounds, this held not true for the terminal olefins when compared to performic acid in chloroform. This system was the only one able to epoxidize terminal olefins in a reasonable time and was therefore chosen for the aspired sample preparation procedure.

### 3B Elucidation of MOAH Chromatograms by GC-MS

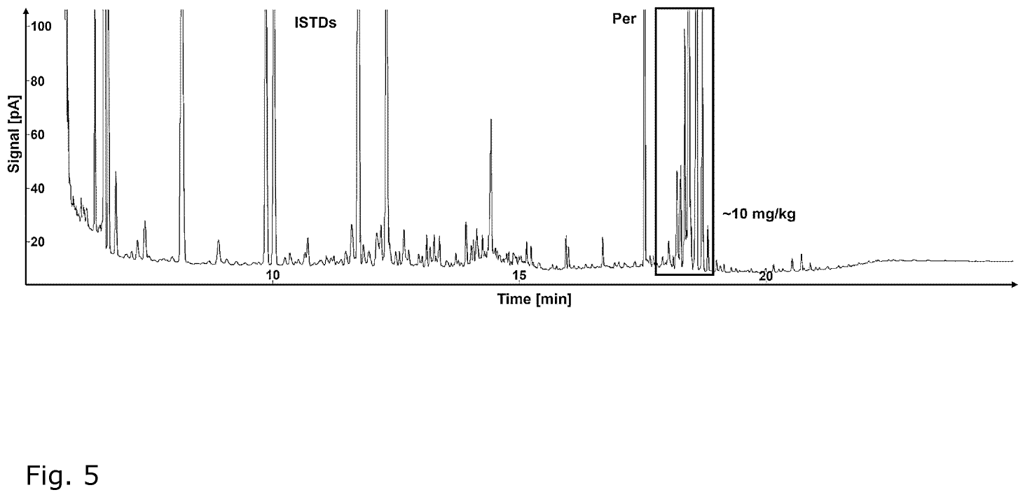

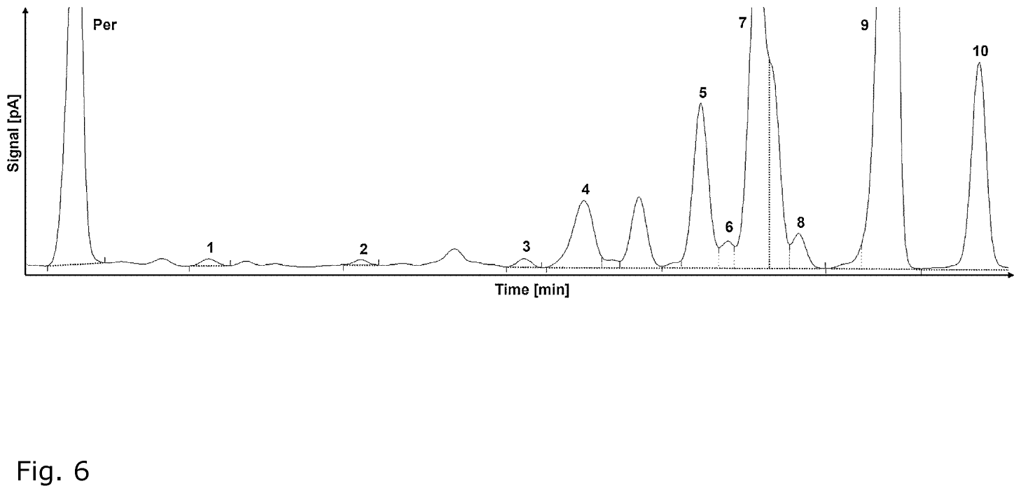

Next, chromatographic signals were investigated after epoxidation. MOAH fractions were collected after HPLC separation and subjected to GC-MS analysis. Even though most signals could not be identified safely, a very prominent peak cluster was found in almost all analyzed refined palm oil fractions (highlighted in FIG. 5 and magnified in FIG. 6). Interestingly, it was not present in virgin palm oil hinting to formation during the refining process.

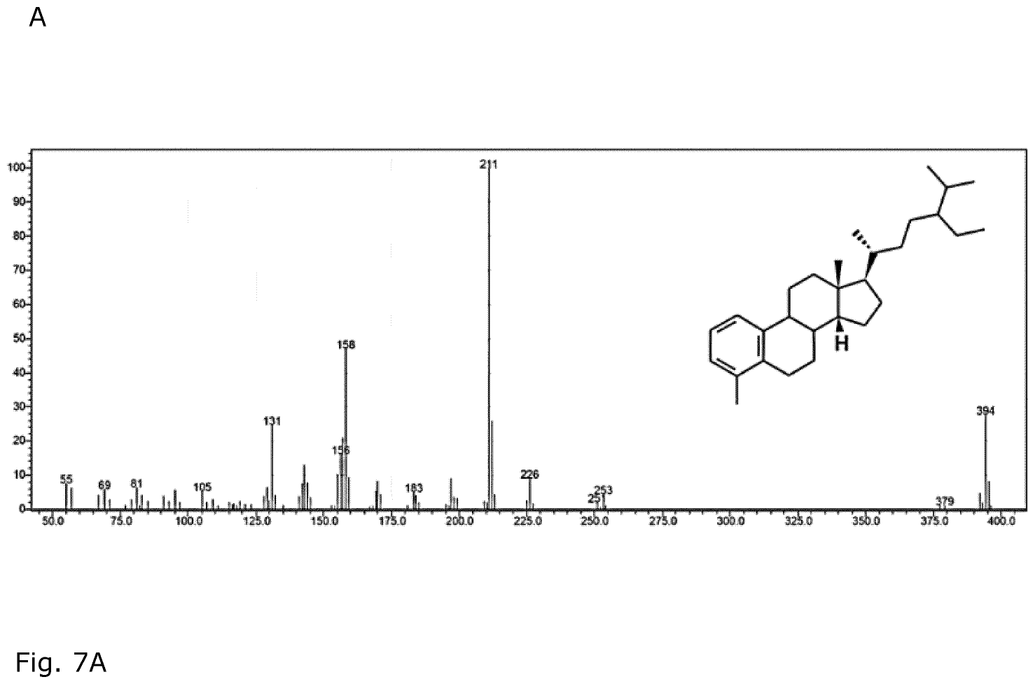

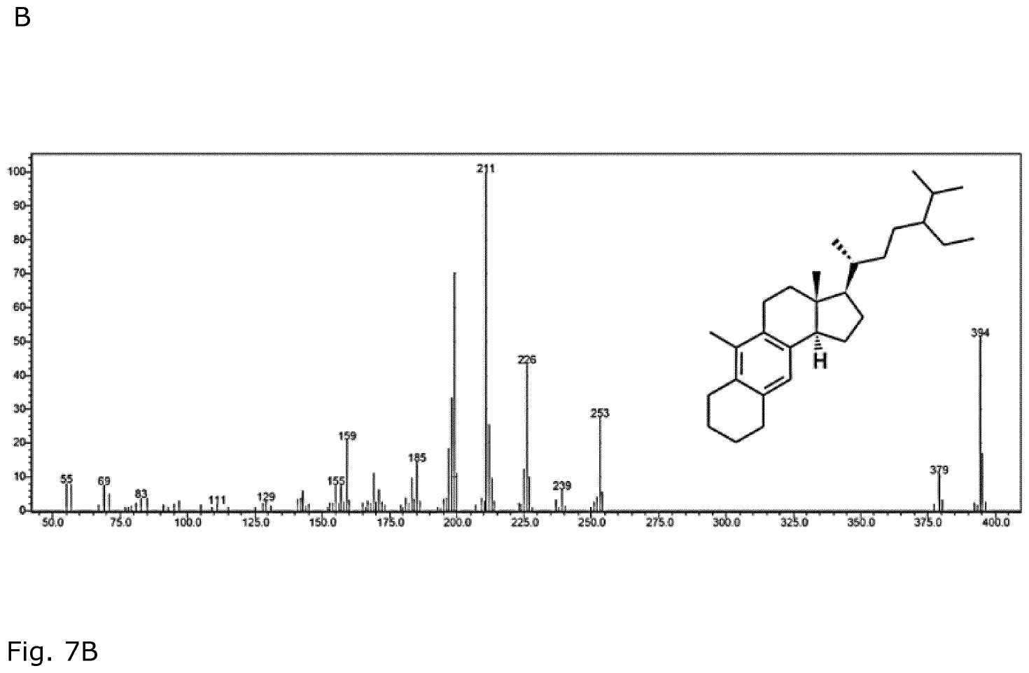

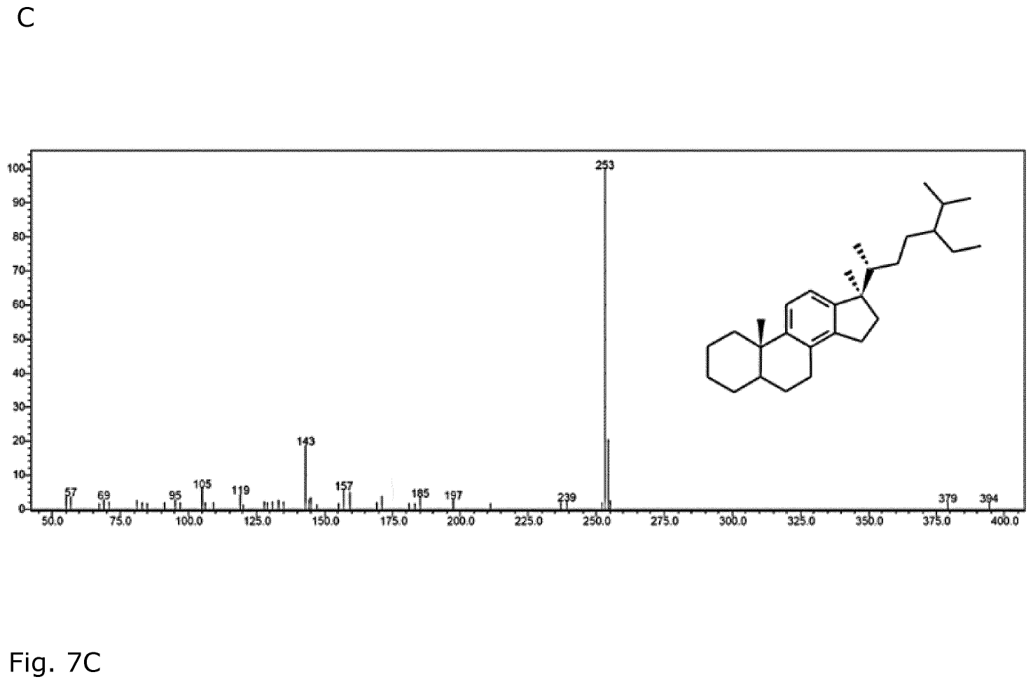

The mass spectra indicated the inheritance of these compounds from phytosterols. The finding of very abundant m/z 211 and m/z 253 hinted to the presence of aromatic steroid compounds. Without wishing to be bound by theory it is proposed that dehydration leads to formation of steratrienes. Subsequent rearrangement of the double bonds with aromatization of one ring moiety could lead to monocyclic aromatic steroidal hydrocarbons (see FIG. 7A-C). Since these compounds share the same aromatic structures as a majority of MOAH, it is proposed that they cannot removed by epoxidation and need to be distinguished from the rest in other ways such as by careful visual inspection of the LC-GC-FID chromatograms of by GC×GC.

Per definition, classification of MOAH is solely based on their chromatographic elution profile on bare silica gel and the convention to collect the eluate containing highly alkylated MACs and perylene. The following substance classes typically found in (non-refined) mineral oils are eluted within this fraction:

(Partially hydrogenated) monocyclic, bicyclic, tricyclic, and polycyclic aromatic compounds (MACs, BACs, TACs, and PACs) with a varying degree of alkylation, aromatic heterocycles containing sulfur or oxygen, e.g., PASHs or PAOHs (nitrogen-containing heterocycles, i.e., PANHs, are not eluted in the same window), MACs and to some extent BACs are the most prominent substance classes found in MOAH. Higher conjugated aromatic systems are found to much lesser extent.

For a simple test which substances are affected by epoxidation, a model mixture was composed containing the MOH ISTDs, EPA PAHs, dibenzofurane, and dibenzothiophene. This mixture was subjected to epoxidation. Additionally, two commercially available mineral oils were epoxidized. In Table 2, the results are summarized.

TABLE 2
Half-life times of MOAH model substances subjected
to epoxidation with performic acid

| | t_{1/2} [min] | |
|---|---|---|
| | n-Hexane | CHCl₃ |
| MACs | | |
| n-Pentylbenzene | ∞ | ∞ |
| TBB | ∞ | 91 |
| BACs | | |
| Naphthalene | 240 | 232 |
| 1-Methylnaphthalene | 152 | 44 |
| 2-Methylnaphthalene | 162 | 35 |
| TACs | | |
| Acenaphthene (1,8-Ethylenenaphthalene) | 109 | 7.4 |
| Acenaphthylene (Cyclopenta[de]naphthalene) | 1.9 | <1.0 |
| Fluorene^{a} | ∞ | ∞ |
| Dibenzofurane^{b} | ∞ | ∞ |
| Dibenzothiophene^{b} | 2.5 | 1.3 |
| Anthracene | 13 | 2.5 |
| Phenanthrene | 216 | 82 |
| PACs | | |
| Fluoranthene | 661 | 229 |
| Pyrene | 62 | 7.4 |
| Benz[a]anthracene | 48 | 3.9 |
| Chrysene | 240 | 34 |
| Benzo[b]fluoranthene, Benzo[k]fluoranthene^{c} | 467 | 40 |
| Benzo[a]pyrene | 15 | <1.0 |
| Perylene | 9.9 | 1.1 |
| Indeno[1,2,3-cd]pyrene | 198 | 19 |
| Dibenz[a,h]anthracene | 127 | 6.7 |
| Benzo[ghi]perylene | 100 | 5.5 |

^{a}contains two separated benzene moieties responsible for its stability
^{b}sp² re-hybridization at the heteroatom leads to a Hückel-aromatic system
^{c}Substances were not separated on the used GC column

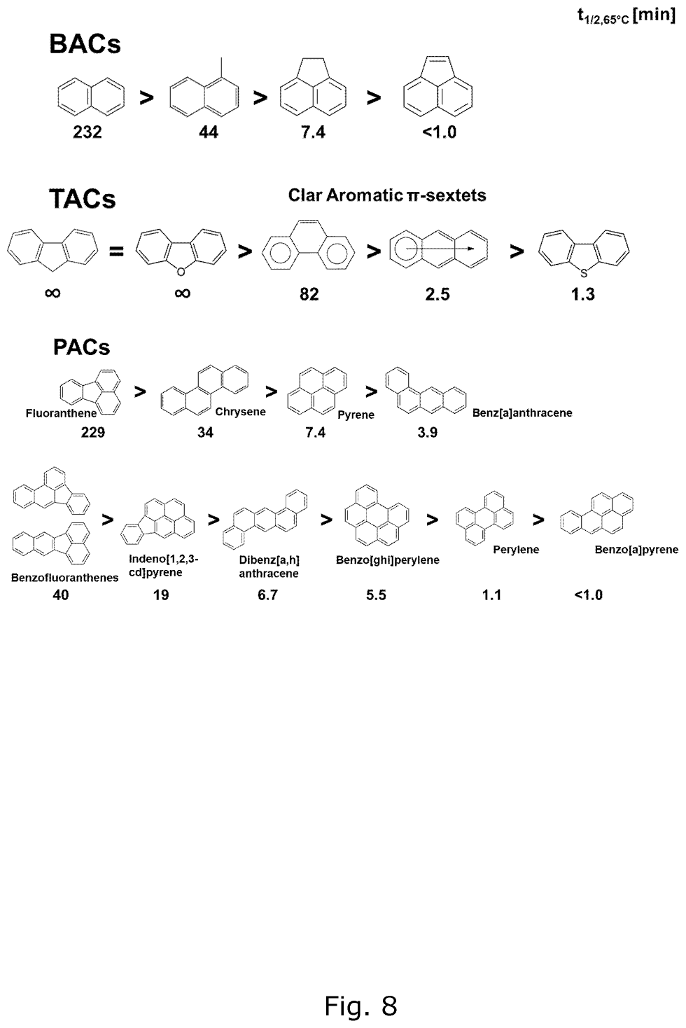

First, it is noteworthy that despite a few exceptions, all compounds exhibit much higher half-life times than olefins (see Table 2 and FIG. 8). MACs and DBF (as representative for PAOHs) are not affected by epoxidation significantly. Even in chloroform without any competing sample matrix, the half-life time of TBB is higher than 90 min. PASHs are oxidized within short time to sulfoxides and eventually sulfones. For BACs, TACs, and PACs, oxidation products such as phthalic acid or quinones were reported in literature. Losses do not strictly correlate with ring sizes or ionization energies but are highly dependent on the used solvent. Not only does chloroform allow for epoxidation of terminal olefins, but it also accelerates oxidation of aromatic substances.

As can be further seen in Table 2 for the class of naphthalenes (BACs), losses for methylated naphthalenes and acenaphthene are higher than for the parent compound indicating that hyperconjugation due to alkylation has significant influence. The high losses of acenaphthylene are caused by its prominent double bond susceptible to oxidation.

In the TAC class, anthracene is lost to much higher extent than phenanthrene.

It can be summarized that the majority of the quantified MOAH content, i.e., MACs and BACs, is mainly unaffected by epoxidation while harsh epoxidation conditions are inevitable to remove olefins otherwise hindering reliable MOAH quantitation.

### Lowering the Limit of Quantitation

One of the easiest ways to improve sensitivity of an analytical method is to increase the sample amount that is analyzed. Obviously, by increasing the sample amount, the amount of unwanted sample by-products (or matrix) is increased as well. For the analytics of plain hydrocarbons, saponification is a valuable tool to remove the vast majority of matrix. Typically, the unsaponifiable matter of typical edible oils or fats accounts for only 1-2% of the total weight.

While in the initial stages of MOSH/MOAH analytics, the HPLC clean-up on bare silica gel served to remove the matrix, this approach becomes impractical when the sample amount is increased beyond the limits of the used HPLC column. Choosing a bigger HPLC column leads to problems regarding the transfer volume into GC. Therefore, saponification is the easiest solution to tackle this obstacle even if it leads to a more complex sample preparation. Conveniently, this step can be automated.

Since EN16995:2017 made use of 20 mg of sample amount and was validated with an LOQ of 10 mg/kg, it is reasonable to assume that roughly 200 mg are needed to obtain 1 mg/kg (if no other interferences hinder quantitation). The DGF standard method makes use only of 100 mg of sample to facilitate manual work in the laboratory. In case of very low MOAH contents, a second evaporation step is mandatory to increase the sample amount to approximately 200-300 mg. In the current work, the scope was to develop methods to work with at least 200-300 mg sample size, preferably 400 mg.

Typically, an excess of potassium hydroxide in ethanol is used to saponify mainly triglycerides at elevated temperatures. Other hydroxides or alcohols often result in formation of insoluble fatty acid salts. Afterward, the unsaponifiable matter is extracted with non-polar solvents such as n-hexane. This approach is applicable for MOSH/MOAH detection as well.

Low recovery of the ISTDs after saponification and extraction of the unsaponifiable matter was reported in the past. Apparently, high amounts of soaps formed during saponification can encapsulate parts of the ISTDs, particularly BACs. Although the influence on MOAH is not described in literature, it is fair to assume that similar compounds are partially lost as well. Own tests revealed losses as high as 20% for methylnaphthalenes while the other standards were fully recovered. Appending a second n-hexane extraction solved this problem.

### Method Design

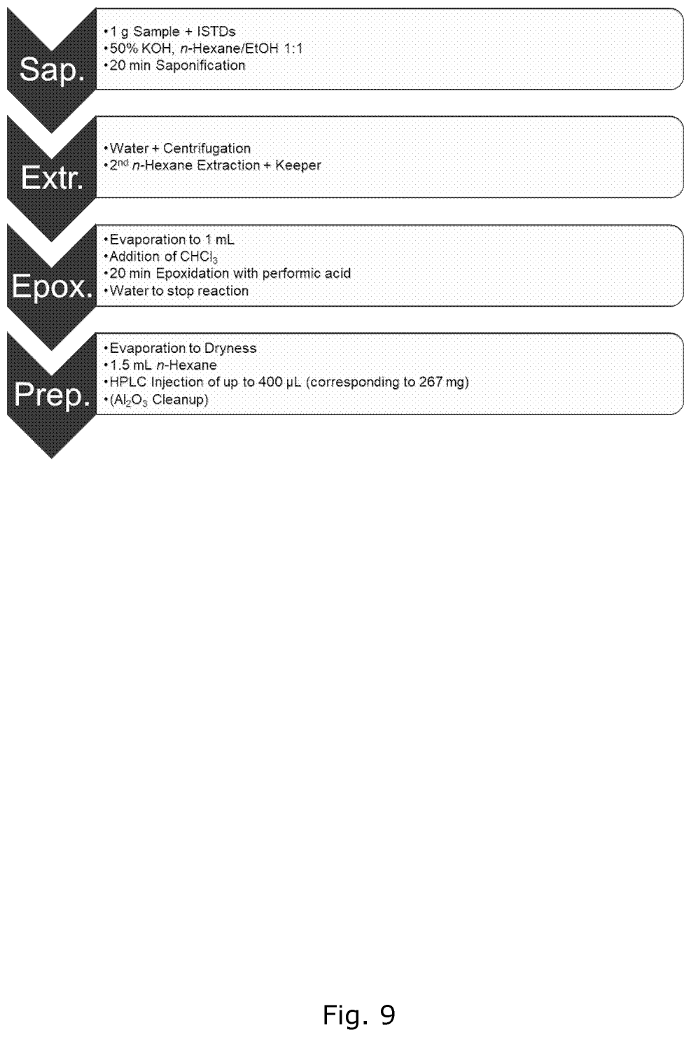

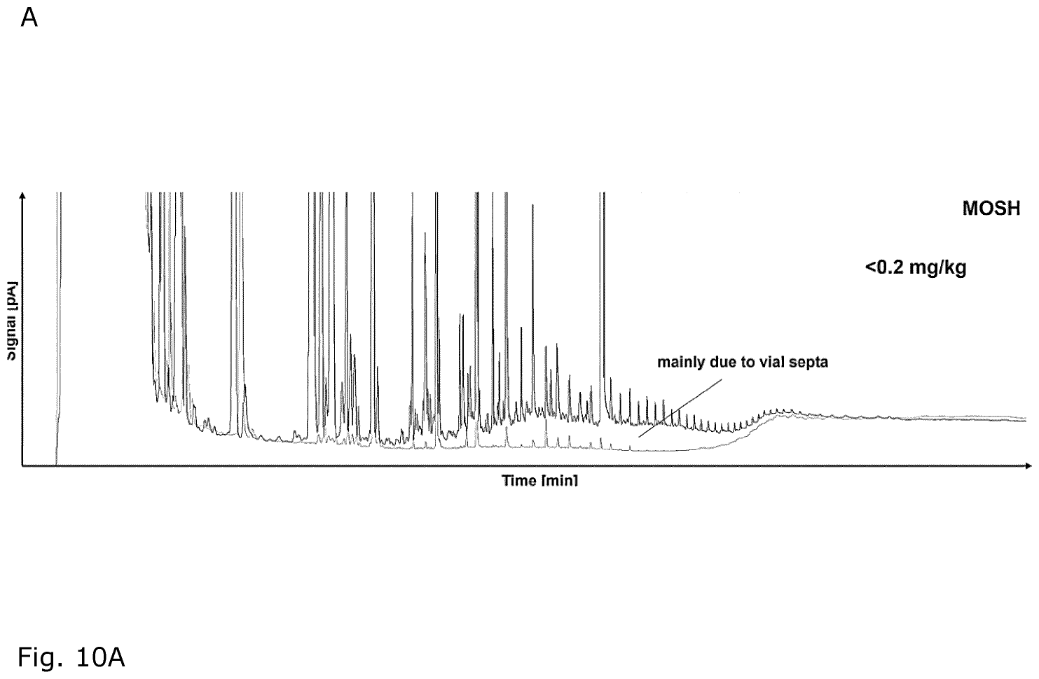

Since epoxidation with performic acid in chloroform showed the best removal of interferences, it was used in conjunction with saponification. The solvent switching prior to HPLC injection was not considered as a problem since all processes were completely automated on the used autosampler. This rendered user-related issues during evaporation steps irrelevant. In FIG. 9 the whole process is presented.

To assist with hexane removal and solvent switch to chloroform, a keeper solvent was added in advance. The traditionally used keeper bis(2-ethylhexyl) maleate could not be used, because it disturbs epoxidation due to its olefinic double bond. The corresponding sebacate, however, was found to be suited for that purpose.

Manual silica gel column chromatography was omitted, because it was found that epoxidation with performic acid under highly acidic conditions showed three important benefits compared to traditional methods. First, polar sample by-products did not retard epoxidation. Secondly, washing of the n-hexanic phase after saponification was not necessary, because residual fatty acid soaps were neutralized and eventually eliminated by HPLC. Finally, the formed epoxides were quickly cleaved by aqueous acid. The resulting diols exhibit higher retention on bare silica gel. Thus, in absence of triglycerides, the used HPLC column could be used for removal of these compounds making manual column chromatography superfluous for samples where the percentage of unsaponifiable matter is low. Visual inspection of the MOAH chromatograms verified the absence of polar by-products for typical oils and fats. Anyhow, in case of matrix breakthrough due to column aging, flushing with polar eluents was an appropriate way to recover the performance of the HPLC column.

As a final change to the original method, the maximum injection volume into HPLC was increased from 100 to 400 μL by using an enlarged sample loop and slight adjustments on the HPLC and GC transfer conditions. This change rendered the final evaporation step during sample preparation more robust, because the otherwise needed volume of 375 μL could be increased to 1500 μL making it possible to use a 10-mL autosampler vial. Otherwise, either special vials with conical inserts would have been needed or the evaporation would have to be done in two steps with intermediate liquid transfer to a smaller vial size.

### Method Validation

The proposed sample preparation involving saponification, enrichment and epoxidation was tested in terms of reproducibility on olive oil from a local supermarket. Eight measurements in three weeks gave a measured value for MOSH and MOAH of 14.9±1.0 mg/kg and 2.1±0.1 mg/kg, respectively. Relative standard deviation under reproducibility conditions was below 7% fully compliant with the accepted value of 25% according to latest collaborative trials.

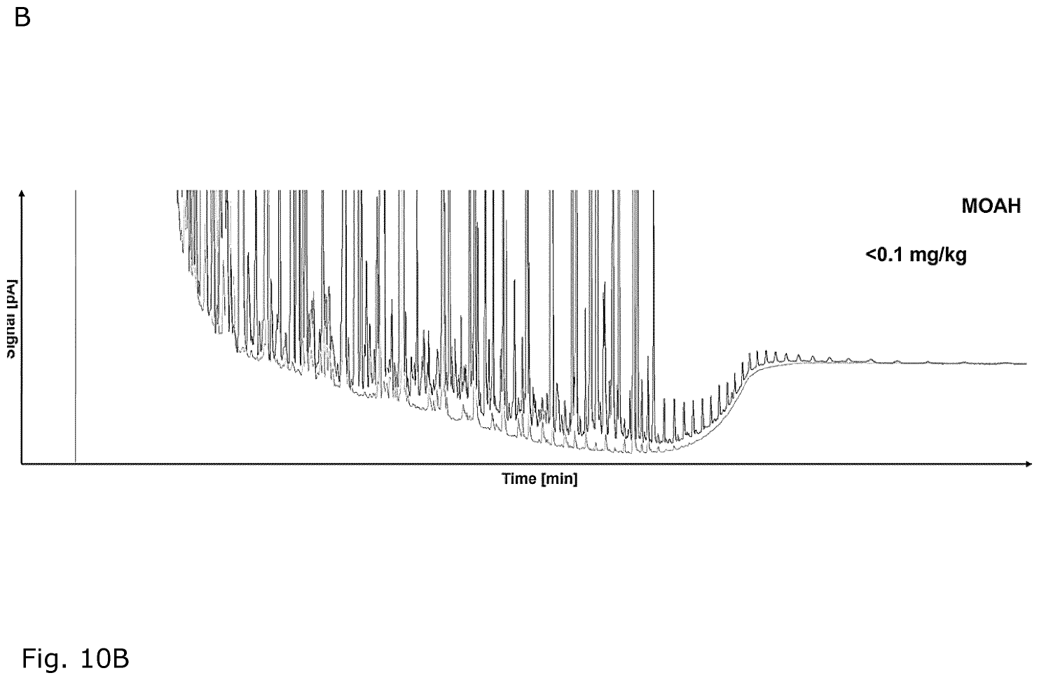

According to the DGF standard method, blank levels should be less than ⅓ of the aspired LOQ. Subjection of reagent blanks to the proposed method revealed blank values for MOSH and MOAH of <0.2 mg/kg and <0.1 mg/kg, respectively (see FIGS. 10A and 10B). Thus, blank values are of no relevance for an aspired LOQ of at least 1 mg/kg.

Trueness and recovery experiments were conducted on well-known samples previously used in a collaborative trial in scope of the DGF standard method development. In Table 3, the obtained results are summarized showing that within a z-score range of ±2 acceptable comparison is generally given.

TABLE 3
Comparison of collaborative trial MOAH results,
and values obtained by the automated workflow

| | | Sun- | Rapeseed | Olive | Sunflower | Palm |
|---|---|---|---|---|---|---|
| | Cocoa | flower | Oil | Oil | Oil | Oil |
| | Butter | Oil | Spiked | Spiked | Spiked | Spiked |
| MOSH ≤ | 87.2 | 5.0 | 3.1 | 13.3 | 9.7 | 22.1 |
| C50 | | | | | | |
| [mg/kg] | | | | | | |
| CT Mean | 98.9 | 2.9 | 2.0 | 9.6 | 7.9 | 22.7 |
| [mg/kg] | | | | | | |
| z-Score | −0.6 | 4.9 | 2.8 | 2.2 | 1.5 | −0.1 |
| MOAH ≤ | 34.2 | 0.6 | 0.3 | 1.3 | 1.3 | 2.8 |
| C50 | | | | | | |
| [mg/kg] | | | | | | |
| CT Mean | 43.2 | 0.6 | 0.5 | 1.4 | 1.2 | 3.8 |
| [mg/kg] | | | | | | |
| z-Score | −1.7 | −0.4 | −1.7 | −0.3 | 0.2 | −1.0 |

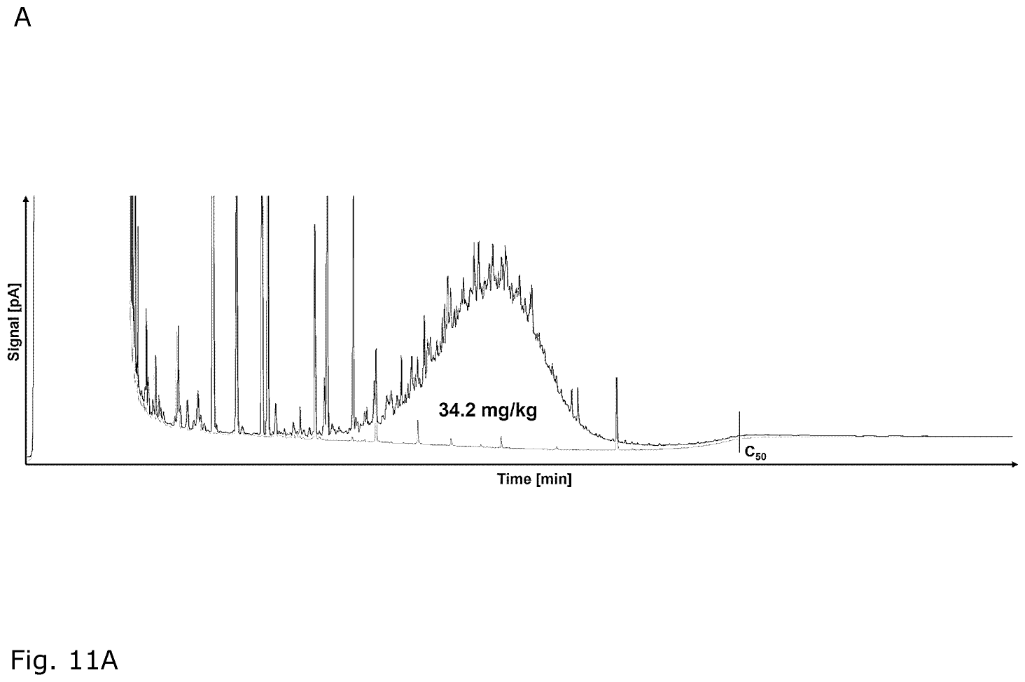

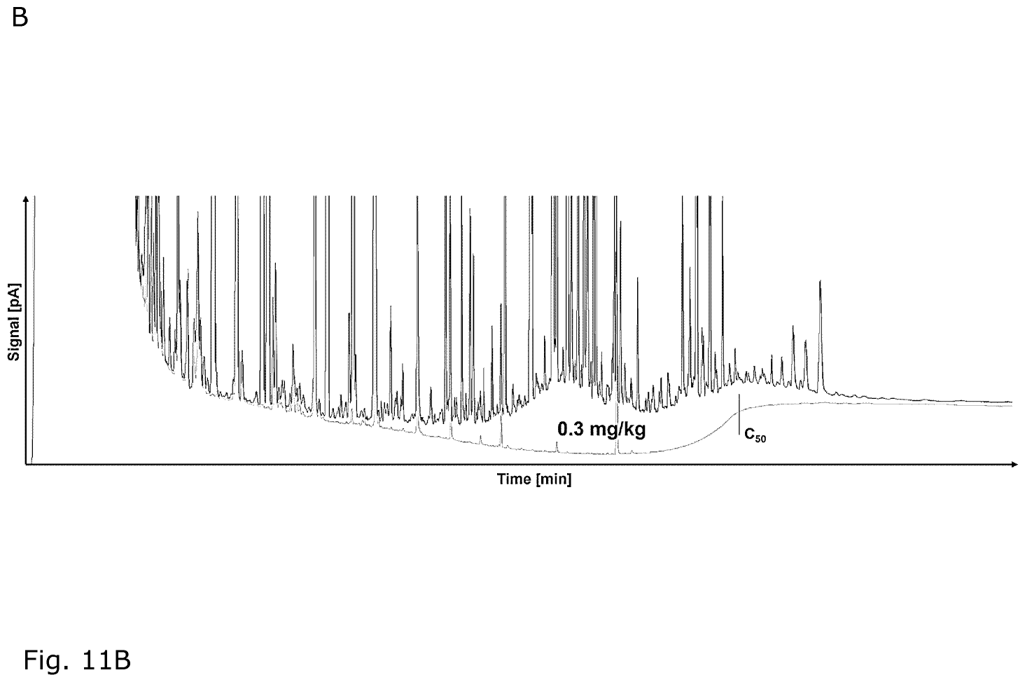

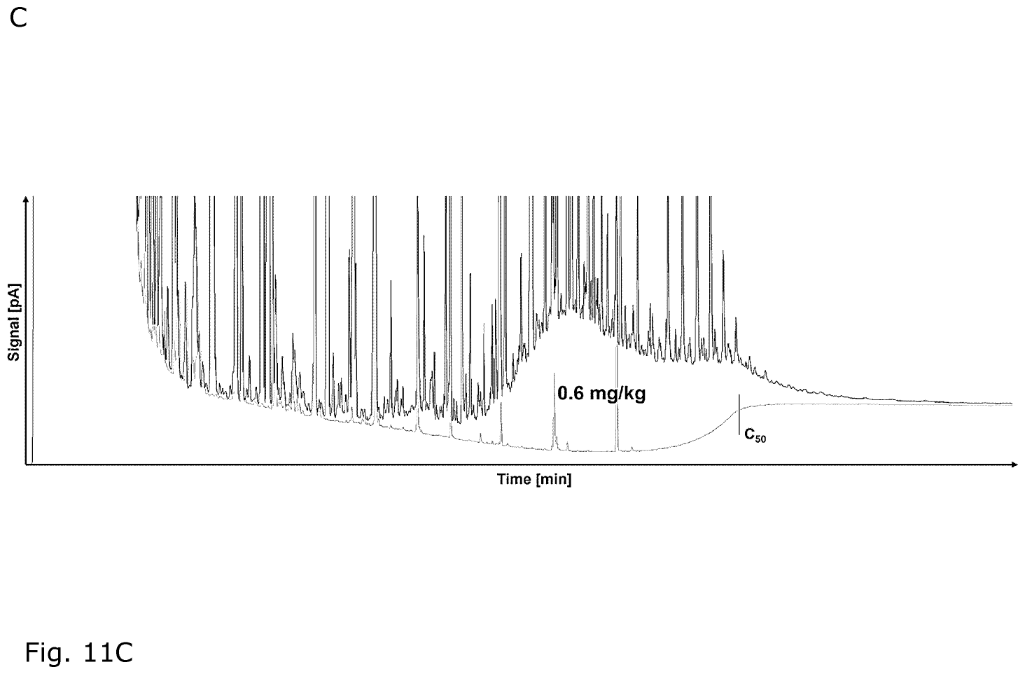

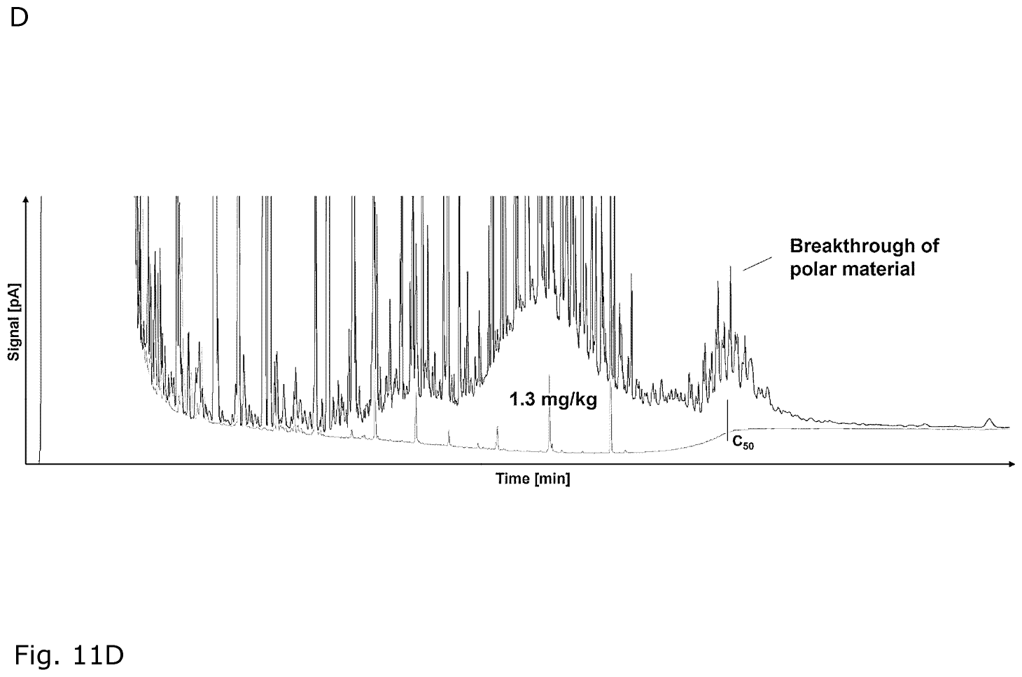

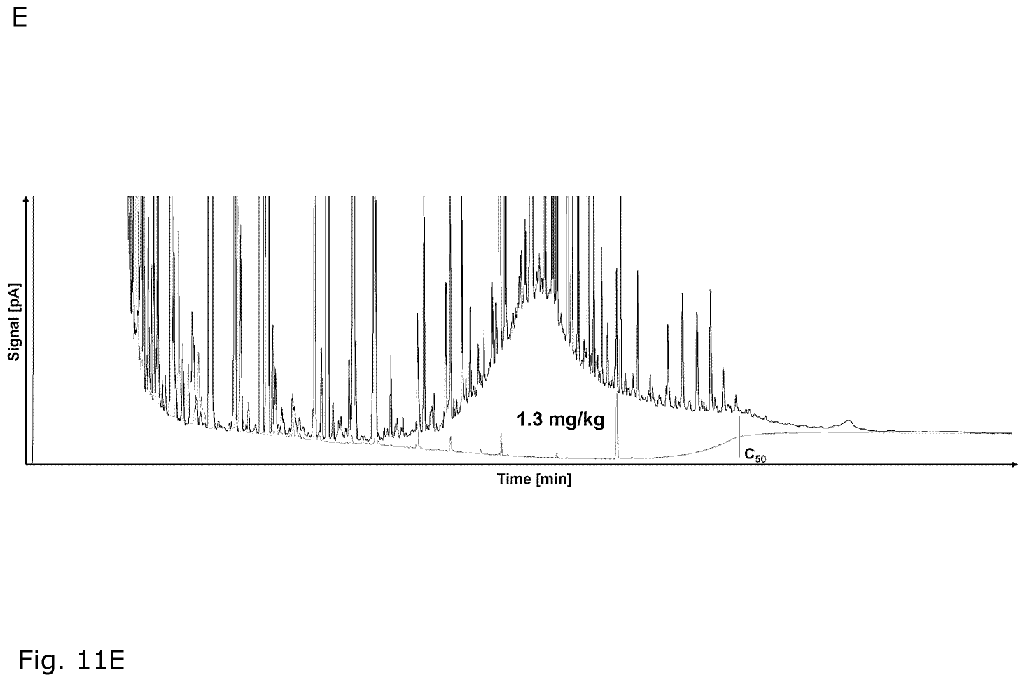

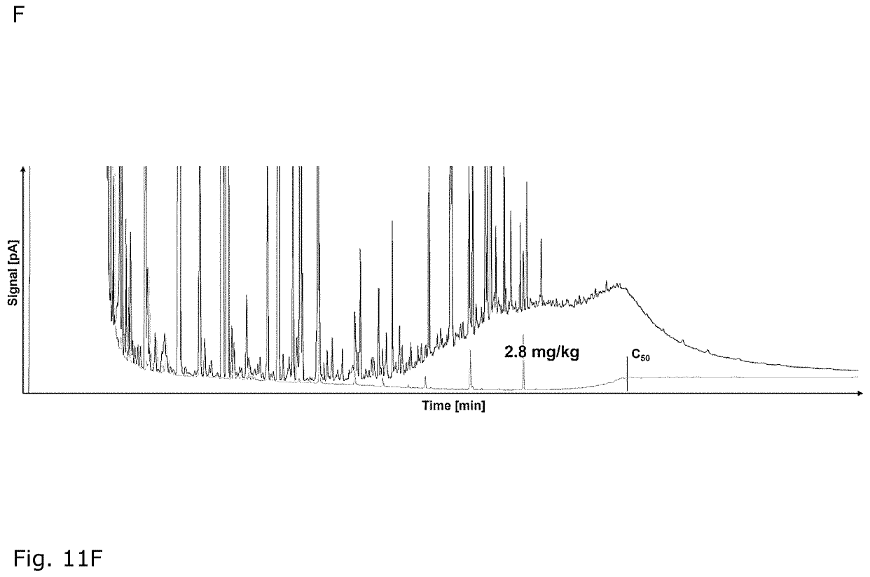

However, for cocoa butter, spiked rapeseed, and palm oil rather negative MOAH z-scores were obtained. Whether this was related to unintended losses of MOAH or overestimation of MOAH by the collaborative trial's participants could not be fully clarified. The corresponding chromatograms are shown in FIGS. 11 A-F. The MOAH results for sunflower and rapeseed oils below the aspired LOQ indicate how important it is to inject as much sample amount as possible with lowest achievable contaminations. Otherwise, reliable integration becomes impossible.

For MOSH, samples containing high amounts of biogenic n-alkanes show z-scores out of the aspired range. This can be attributed to variances in the efficiency of the aluminium oxide clean-up.

### CONCLUSION

The results show that removal of persistent biogenic interferences should be favored over milder reaction conditions for epoxidation. Epoxidation with performic acid in chloroform proved to be the most effective reagent to remove biogenic rr-electron deficient olefins and marks an important achievement in MOSH and MOAH sample preparation. While MOAH losses of TACs and PACs cannot be entirely prevented, quantitatively they are often irrelevant due to the low abundance of these compounds in refined mineral oils. Major classes, such as MACs and BACs, are affected by epoxidation to much lesser extent.

Paired with a streamlined workflow involving saponification, enrichment, and increase of injection volume into HPLC, a fully automated method was established. Quantitative results for collaborative trial samples verified the trueness of the proposed method making it amenable to the analysis of edible oils and fats in routine environments.

1. A method for preparing a sample for analysing mineral oil contamination in a foodstuff, wherein the method is at least partially automated, the method comprising:
(i) contacting and dissolving, or extracting, the foodstuff with an excess of a solution of a hydrocarbon solvent, and optionally an alcohol, at a temperature between 20° C. and the boiling point of the solvent mixture;
(ii) cooling, and if an alcohol was used in the previous step, initiation of phase separation by addition of water or a mixture of water and the alcohol;
(iii) contacting an organic phase comprising the dissolved or extracted foodstuff with an excess of at least 30% hydrogen peroxide and a compound of the general formula (I)
R¹—COOH (I)
in which R¹ represents H or the partial general formula (II)
(R²)(R³)C(R⁴)— (II)
wherein R², R³ and R⁴, independently of one another, represent H, F, Cl, OH or methyl, in the presence of a catalytic amount of a mineral acid at a temperature of about 20° C. to about 70° C. for about 5 min to about 2 h;
(iv) partitioning the solution by adding an excess of water to provide an aqueous phase and a reacted organic phase, and
(v) separating the reacted organic phase for the analysis of mineral oil contamination in the foodstuff; and
(vi) optionally analysing the sample.

2. The method of claim 1, wherein prior to the epoxidation step (iii) the organic phase is evaporated and an amount of halogenated aliphatic or aromatic hydrocarbon is added, wherein the amount is an amount necessary to dissolve the sample; or
wherein the epoxidation step (iii) is conducted in the presence of a halogenated aliphatic or aromatic hydrocarbon.

3. The method of claim 1 or 2, wherein the method further comprises, after step (iii), evaporating the organic phase and exchanging the solvent for n-hexane.

4. The method of any one of claims 1 to 3 wherein the hydrocarbon solvent is a hexane or a pentane, or a combination thereof

5. The method of any one of claims 1 to 4 wherein the hydrocarbon solvent is solvent is selected from n-hexane, n-pentane, iso-pentane, iso-hexane, or a combination thereof

6. The method of any one of claims 1 to 5 wherein the hydrocarbon solvent is n-hexane.

7. The method of any one of claims 1 to 6 wherein the alcohol is ethanol.

8. The method of any one of claims 1 to 7, wherein the method is fully automated.

9. The method according to any one of claims 1 to 8, wherein the foodstuff comprises alkaline hydrolyzable constituents in the organic phase and following step (ii) and prior to step (iii) the alkaline hydrolyzable constituents are saponified by:
contacting the dissolved or extracted foodstuff with an excess of a concentrated aqueous alkali metal hydroxide solution or other strong base above about 20° C. to about 70° C., and
extracting the non-saponified constituents by adding the hydrocarbon solvent; and
optionally repeating the extraction with the hydrocarbon solvent one or more times.

10. The method according to any one of claims 1 to 9, wherein the foodstuff comprises acidic hydrolyzable constituents in the organic phase and following step (ii) and prior to step (iii) the acid hydrolyzable constituents are hydrolyzed by:
contacting the dissolved or extracted foodstuff with an excess of a concentrated aqueous hydrochloric acid solution or other strong acid at about 20° C. to about 70° C.; and
extracting the non-hydrolyzed constituents by adding a hydrocarbon solvent; and
optionally repeating the extraction with the hydrocarbon solvent one or more times.

11. The method according to any one of claims 1 to 10, wherein, the food stuff is a dried foodstuff or is a foodstuff present in aqueous phase and, prior to step (i) and prior to step (ii) the mineral oil impurities are extracted from the foodstuff by
contacting the foodstuff with an excess of an organic water-insoluble solvent; and
separating the solvent comprising extracted mineral oil impurities from the foodstuff using suitable means.

12. The method according to any one of claims 1 to 11, wherein the dissolution and/or extraction of the mineral oil components in the food is carried out with an excess of a solution of hydrocarbon solvent and an alcohol, wherein the vol:vol ratio of the hydrocarbon solvent to ethanol is 1:x wherein x is a number from 0 to 10 at a temperature or from about 20° C. to the boiling point of the solvent mixture.

13. The method according to claim 9, wherein the saponification of the organic phase is carried out with an excess of a concentrated alkali metal hydroxide solution in the form of sodium hydroxide solution and/or potassium hydroxide solution at from about 20° C. to about 70° C.

14. The method according to any one of claims 1 to 13, wherein the halogenated hydrocarbon chloroform is added to the organic phase before the epoxidation, step (iii), is started.

15. The method according to any one of claims 1 to 14, wherein the epoxidation, step (iii) comprises
contacting the organic phase with a mixture of 50% hydrogen peroxide and concentrated formic acid and/or acetic acid in the presence of a catalytic amount of concentrated phosphoric acid and/or sulfuric acid at a temperature of from about 20° C. to about 70° C. for about 10 min to about 60 min.

16. A method according to any one of the preceding claims, wherein the foodstuff is a substance or product intended to be or reasonably expected to be ingested by humans in a processed, partially processed or unprocessed state and in particular selected from natural fats, protein-containing foods, carbohydrate-rich foods, alcohol-containing foods, alkaloid-containing foods, vegetables and vegetable products, fruits and fruit products, spices or herbs, drinking water, soft drinks, functional foods, food supplements, dietary foods, novel foods, vitamins, minerals, enzymes, lipids, amino acids, additives or mixtures of the aforementioned substances and/or products of the food classes.

17. The method according to claim 16, wherein the natural fat is a fruit pulp fat or seed fat or an animal fat, or a mixture of the aforementioned fats, preferably an edible oil or fat.

18. The method according to claim 17, wherein fat-containing food is milk or a milk product, chocolate, chocolate products, cocoa, margarine products, mixed fat products, infant formula and follow-on formula, or a mixture of the aforementioned products.

19. The method according to any one of claims 1 to 18 wherein step (vi) is performed by a coupled liquid chromatogram and gas chromatogram, preferably equipped with a FID detector (LC-GC-FID).

20. A system for carrying out the partially automated methods for analysing mineral oil contaminants in foodstuff comprising:
a receptacle for a sample of foodstuff for analysis of its mineral oil content, the receptacle in fluid communication with a first well, a second well, a third well and a fourth well;
the first well adapted for the introduction into the receptacle of a hydrocarbon solvent and optionally an alcohol at a temperature of from about 20° C. to the boiling point of the solvent mixture,
the second well adapted for the introduction to the receptacle of an excess of at least 30% hydrogen peroxide
a third well adapted for the introduction to the receptacle of a compound of the general formula (I):
R¹—COOH (I)
in which R¹ represents H or the partial general formula (II)
(R²)(R³)C(R⁴)— (II)
wherein R², R³ and R⁴, independently of one another, are H, F, Cl, OH or methyl, preferably in the presence of a catalytic amount of a mineral acid at a temperature of from about 20° C. to about 70° C.
the fourth well adapted to introduce water into the receptacle
a separator to separate a reacted organic phase from the receptacle and transporting the hydrocarbon phase to an analyser
a controller for controlling the addition to the receptacle of the solvent contained in each of the first, second, third and fourth wells, and also for controlling the temperature of the receptacle; and
optionally the analyser capable of detecting the mineral oil in the reacted organic phase.

21. The system of any one of claim 20 wherein the controller is adapted to carry out the method of any one of claims 1 to 19.
